(19) United States
(12) Patent Application Publication
(10) Pub. No.: US 2024/0226097 A1
(43) Pub. Date: Jul. 11, 2024

(54) METHODS OF CANCER TREATMENT USING A COMBINATION OF BTK INHIBITORS AND PI3 KINASE INHIBITORS

(71) Applicants: BeiGene, Ltd., Grand Cayman (KY); BeiGene Switzerland GmbH, Basel (CH)

(72) Inventors: Xiaomin SONG, Beijing (CN); Xiao YANG, Beijing (CN); Nan HU, Beijing (CN); Yuan LIU, Beijing (CN); Jing LI, Beijing (CN); Zhiwei WANG, Beijing (CN)

(73) Assignees: BeiGene, Ltd., Grand Cayman (KY); BeiGene Switzerland GmbH, Basel (CH)

(21) Appl. No.: 18/600,385

(22) Filed: Mar. 8, 2024

Related U.S. Application Data
(63) Continuation of application No. PCT/CN2022/118351, filed on Sep. 13, 2022.

(30) Foreign Application Priority Data
Sep. 14, 2021 (WO) PCT/CN2021/118152
Aug. 26, 2022 (WO) PCT/CN2022/115148

(51) Int. Cl. A61K 31/4985; A61P 35/00

(52) U.S. Cl. CPC A61K 31/4985; A61P 35/00

(57) ABSTRACT

Disclosed herein is a method for the treatment or delay of progression of cancer in a subject, comprising administering to the subject in need thereof a BTK inhibitor, for example, (S)-7-(1-acryloylpiperidin-4-yl)-2-(4-phenoxyphenyl)-4,5,6,7-tetra-hydropyrazolo[1,5-a]pyrimidine-3-carboxamide or a pharmaceutically acceptable salt thereof, in combination with a PI3Kδ inhibitor or a pharmaceutically acceptable salt thereof.

### CROSS-REFERENCE TO RELATED APPLICATIONS

This application is a continuation of International Patent Application No. PCT/CN2022/118351, filed Sep. 13, 2022, which claims priority from International Patent Application Nos. PCT/CN2021/118152, filed Sep. 14, 2021, and International Patent Application No. PCT/CN2022/115148, Aug. 26, 2022. The contents of these applications are incorporated herein by reference in their entirety.

### FIELD OF THE DISCLOSURE

Disclosed herein are methods for the treatment, delay of progression or prevention of cancer in a subject, comprising administering to the subject in need thereof a Bruton's tyrosine kinase (BTK) inhibitor (e.g., (S)-7-(1-acryloylpiperidin-4-yl)-2-(4-phenoxyphenyl)-4,5,6,7-tetrahydropyrazolo-[1,5-a]pyrimidine-3-carboxamide or a pharmaceutically acceptable salt thereof) in combination with a phosphoinositide 3-kinase 6 (PI3K6) inhibitor or a pharmaceutically acceptable salt thereof. Disclosed herein is also a pharmaceutical combination comprising a BTK inhibitor (e.g., (S)-7-(1-acryloylpiperidin-4-yl)-2-(4-phenoxyphenyl)-4,5,6,7-tetra-hydropyrazolo[1,5-a]pyrimidine-3-carboxamide or a pharmaceutically acceptable salt thereof) and a PI3K6 inhibitor or a pharmaceutically acceptable salt thereof and methods of treatment thereof.

### BACKGROUND OF THE DISCLOSURE

Bruton's tyrosine kinase (BTK) belongs to the Tec family of cytoplasmic tyrosine kinases, which is the second largest family of non-receptor kinases in humans (Vetrie et al., Nature 361: 226-233, 1993; Bradshaw, Cell Signal. 22: 1175-84, 2010). It is expressed in all cell lineages of the hematopoietic system, except for T cells and it is localized in bone marrow, spleen and lymph node tissue (Smith et al., J. Immunol. 152: 557-565, 1994). Inactivating mutations in the gene encoding BTK result in X-linked agammaglobulinemia (XLA) in humans and X-linked immunodeficiency (XID) in mice (Conley et al., Annu. Rev. Immunol. 27: 199-227, 2009). Both diseases are characterized by dramatic defects in B cell development and function, suggesting the essential role of BTK for B cell development and function. In contrast, constitutive activation of BTK in B cells results in the accumulation of autoreactive plasma cells (Kersseboom et al., Eur J Immunol. 40:2643-2654, 2010). BTK is activated by upstream Src-family kinases in the BCR signaling pathway. Once activated, BTK, in turn, phosphorylates phospholipase-Cγ (PLCγ), leading to Ca²⁺ mobilization and activation of NF-κB and MAP kinase pathways. These proximal signaling events promote expression of genes involved in proliferation and survival (Humphries et al., J. Biol. Chem. 279: 37651, 2004). In addition to its essential regulatory role as downstream of BCR, BTK activity also plays a critical role in FcR signaling. Signaling via FcRγ associated receptors also promotes BTK-dependent proinflammatory cytokine production by cells such as macrophages (Di Paolo et al., Nat. Chem. Biol. 7: 41-50, 2011). BTK is an important target due to its proximal location in the BCR and FcR signaling pathways. Preclinical studies show that BTK deficient mice are resistant to developing collagen-induced arthritis. Moreover, clinical studies of Rituxan, a CD20 antibody which depletes mature B-cells, reveals the key role of B-cells in a number of inflammatory diseases such as rheumatoid arthritis, systemic lupus erythematosus and multiple sclerosis (Gurcan et al., Int. Immunopharmacol. 9: 10-25, 2009). In addition, aberrant activation of BTK plays important role in pathogenesis of B-cell lymphomas indicating that inhibition of BTK is useful in the treatment of hematological malignancies (Davis et al., Nature 463: 88-92, 2010).

Diffuse large B cell lymphoma (DLBCL) is an aggressive form of non-Hodgkin lymphoma with two major subtypes, activated B-cell-like (ABC) and germinal center B-cell-like (GCB) DLBCL (Wilson et al. Nat Med. 2015; 21(8):922-6). PI3K6 has been demonstrated to play a crucial role in driving B cell malignancies such as CLL/SLL and NHL (Do et al., Am J Health Syst Pharm. 2016; 73(8):547-55) and the role of BTK in B cell cancers has been discussed above. Given the low response rates, the short duration of response and the potential for both primary and acquired resistance highlight the unmet medical necessity of a combination therapy of a BTK inhibitor and a PI3K6 inhibitor.

### SUMMARY OF THE DISCLOSURE

WO2014/173289 discloses BTK inhibitors, for example, (S)-7-(1-acryloylpiperidin-4-yl)-2-(4-phenoxyphenyl)-4,5,6,7-tetra-hydropyrazolo[1,5-a]pyrimidine-3-carboxamide (hereinafter BTK-1) for the treatment of cancers with aberrations in the B-cell receptor (BCR) and FcR signaling pathway in which BTK plays important roles. BTK-1 has been demonstrated to have potent and irreversible inhibitory activities against BTK.

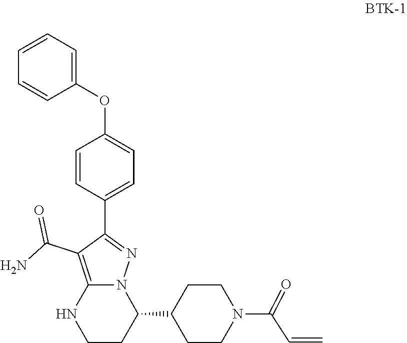 [SMILES: C=CC(=O)N1CCC([C@@H]2CCNc3c(C(N)=O)c(-c4ccc(Oc5ccccc5)cc4)nn32)CC1]

The present disclosure describes a combination of a BTK inhibitor (for example, BTK-1) with a PI3Kδ inhibitor that produces significant inhibition of tumor growth in cancers as compared with the efficacy of each therapeutic as a single agent.

WO2019/047915 discloses a series of imidazo[1,5-a]pyrazine derivative compounds having the following general Formula (I) or a stereoisomer thereof, or a pharmaceutically acceptable salt thereof as PI3Kδ inhibitors, which have demonstrated potent inhibitory activity against phosphatidylinositol-4,5-bisphosphate 3-kinases (PI3K).

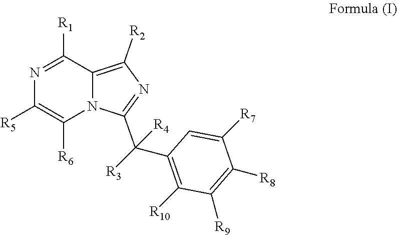

WO2019/047915 discloses useful PI3Kδ inhibitors, for example, (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide (hereinafter Compound 1) for the treatment of cancers.

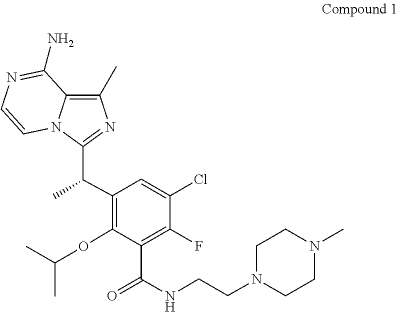 [SMILES: Cc1nc([C@@H](C)c2cc(Cl)c(F)c(C(=O)NCCN3CCN(C)CC3)c2OC(C)C)n2ccnc(N)c12]

In one aspect, disclosed herein is a method for the treatment or delay of progression of cancer in a subject, comprising administering to the subject in need thereof a therapeutically effective amount of (S)-7-(1-acryloylpiperidin-4-yl)-2-(4-phenoxyphenyl)-4,5,6,7-tetra-hydropyrazolo[1,5-a]pyrimidine-3-carboxamide (BTK-1), or a pharmaceutically acceptable salt thereof, in combination with a therapeutically effective amount of PI3Kδ inhibitor or a stereoisomer thereof, or a pharmaceutically acceptable salt thereof.

Also disclosed herein is a pharmaceutical combination for use in the treatment or delay of progression of cancer, comprising (S)-7-(1-acryloylpiperidin-4-yl)-2-(4-phenoxyphenyl)-4,5,6,7-tetra-hydropyrazolo[1,5-a]pyrimidine-3-carboxamide (BTK-1), or a pharmaceutically acceptable salt thereof, in combination with a PI3Kδ inhibitor or a stereoisomer thereof, or a pharmaceutically acceptable salt thereof.

In yet another aspect, disclosed herein is (S)-7-(1-acryloylpiperidin-4-yl)-2-(4-phenoxyphenyl)-4,5,6,7-tetra-hydropyrazolo[1,5-a]pyrimidine-3-carboxamide or a pharmaceutically acceptable salt thereof, for use in the treatment, delay of progression or prevention of cancer in combination with a PI3Kδ inhibitor or a stereoisomer thereof, or a pharmaceutically acceptable salt thereof. In one embodiment, disclosed herein is a PI3Kδ inhibitor or a stereoisomer thereof, or a pharmaceutically acceptable salt thereof, for use in the treatment, delay of progression or prevention of cancer in combination with (S)-7-(1-acryloylpiperidin-4-yl)-2-(4-phenoxyphenyl)-4,5,6,7-tetra-hydropyrazolo[1,5-a]pyrimidine-3-carboxamide, or a pharmaceutically acceptable salt thereof.

The disclosure also provides for a use of a pharmaceutical combination in the manufacture of a medicament for use in the treatment, delay of progression or prevention, said pharmaceutical combination comprising (S)-7-(1-acryloylpiperidin-4-yl)-2-(4-phenoxyphenyl)-4,5,6,7-tetra-hydropyrazolo[1,5-a]pyrimidine-3-carboxamide, or a pharmaceutically acceptable salt thereof, and a PI3Kδ inhibitor or a stereoisomer thereof, or a pharmaceutically acceptable salt thereof.

Articles of manufacture, or “kits” comprising a first container, a second container and a package insert, wherein the first container comprises at least one dose of a medicament comprising (S)-7-(1-acryloylpiperidin-4-yl)-2-(4-phenoxyphenyl)-4,5,6,7-tetra-hydropyrazolo[1,5-a]pyrimidine-3-carboxamide (BTK-1), or a pharmaceutically acceptable salt thereof, the second container comprises at least one dose of a medicament comprising a PI3Kδ inhibitor or a stereoisomer thereof, or a pharmaceutically acceptable salt thereof, and the package insert comprises instructions for treating cancer in a subject using the medicaments is also included.

Provided within the methods of treatment, the cancer is hematologic cancer.

In one embodiment, the hematologic cancer is leukemia, lymphoma, myeloma, non-Hodgkin's lymphoma (NHL), Hodgkin's lymphoma (HL), or B-cell malignancy. In one embodiment, the hematologic cancer is a B-cell malignancy. In another embodiment, the B-cell malignancy is chronic lymphocytic leukemia (CLL), small lymphocytic lymphoma (SLL), follicular lymphoma (FL), mantle cell lymphoma (MCL), marginal zone lymphoma (MZL), Waldenstrom macroglobulinemia (WM), Hairy cell leukemia (HCL), Burkitt's-like leukemia (BL), B cell prolymphocytic leukemia (B-PLL), diffuse large B cell lymphoma (DLBCL), germinal center B-cell diffuse large B-cell lymphoma (GCB-DLBCL), non-germinal center B-cell diffuse large B-cell lymphoma (non-GCB DLBCL), DLBCL with undetermined subtype, primary central nervous system lymphoma (PCNSL), secondary central nervous system lymphoma (SCNSL) of breast or testicular origin, multiple myeloma, extra nodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, lymphomatoid granulomatosis, or a combination thereof.

In one embodiment, the B-cell malignancy is diffuse large B-cell lymphoma (DLBCL). The DLBCL can be activated B-cell diffuse large B-cell lymphoma (ABC-DLBCL), GCB-DLBCL or non-GCB DLBCL. The B-cell malignancy is chronic lymphocytic leukemia (CLL), small lymphocytic lymphoma (SLL), B cell prolymphocytic leukemia (B-PLL), non-CLL/SLL lymphoma, follicular lymphoma (FL), relapse/refractory follicular lymphoma(R/R FL), mantle cell lymphoma (MCL), marginal zone lymphoma (MZL), Waldenstrom's macroglobulinemia (WM), multiple myeloma or a combination thereof. B-cell malignancy also includes resistant B-cell malignancy, wherein the resistant B-cell malignancy is diffuse large B-cell lymphoma (DLBCL), activated B-cell diffuse large B-cell lymphoma (ABC-DLBCL), GCB-DLBCL or non-GCB DLBCL. In another embodiment, the resistant B-cell malignancy is diffuse large B-cell lymphoma (DLBCL), chronic lymphocytic leukemia (CLL), small lymphocytic lymphoma (SLL), B-cell prolymphocytic leukemia (B-PLL), non-CLL/SLL lymphoma, follicular lymphoma (FL), mantle cell lymphoma (MCL), marginal zone lymphoma (MZL), Waldenstrom's macroglobulinemia (WM), multiple myeloma, or a combination thereof.

The B-cell malignancy can also be a metastasized B-cell malignancy. The metastasized B-cell malignancy can be diffuse large B-cell lymphoma (DLBCL), chronic lymphocytic leukemia (CLL), small lymphocytic lymphoma (SLL), B cell prolymphocytic leukemia (B-PLL), non-CLL/SLL lymphoma, follicular lymphoma (FL), relapse/refractory follicular lymphoma(R/R FL), mantle cell lymphoma (MCL), marginal zone lymphoma (MZL), Waldenstrom's macroglobulinemia (WM), multiple myeloma or a combination thereof.

In another embodiment, the cancer is advanced solid tumor.

In another embodiment, the cancer is a sarcoma, or carcinoma. The cancer is bile duct cancer (i.e., cholangiocarcinoma); bladder cancer; breast cancer; cervical cancer; colon cancer; esophageal cancer; ocular cancer; fallopian tube cancer; gastroenterological cancer; kidney cancer; liver cancer; lung cancer; medulloblastoma; melanoma; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; cancer of the head and neck, vaginal cancer; vulvar cancer; or a combination thereof.

The disclosure also provides for the methods of treatment if the cancer is a resistant cancer. The resistant cancer is bile duct cancer (i.e., cholangiocarcinoma); bladder cancer; breast cancer; cervical cancer; colon cancer; esophageal cancer; ocular cancer; fallopian tube cancer; gastroenterological cancer; kidney cancer; liver cancer; lung cancer; medulloblastoma; melanoma; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; cancer of the head and neck, vaginal cancer; vulvar cancer; or a combination thereof.

In another embodiment, the cancer is a metastasized cancer, wherein the metastasized cancer is bile duct cancer (i.e., cholangiocarcinoma); bladder cancer; breast cancer; cervical cancer; colon cancer; esophageal cancer; ocular cancer; fallopian tube cancer; gastroenterological cancer; kidney cancer; liver cancer; lung cancer; medulloblastoma; melanoma; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; cancer of the head and neck, vaginal cancer; vulvar cancer; or a combination thereof.

In the embodiments described above, the PI3Kδ inhibitor is Idelalisib, Copanlisib, Duvelisib, Umbralisib, Leniolisib, Parsaclisib, AMG-319, ME-401, Tenalisib, Linperlisib, Seletalisib, Nemiralisib, KA-2237, SF-1126, HMPL-689, ACP-319, SHC-014748M, AZD-8154, PI3065 or a compound of Formula (I) or a pharmaceutically acceptable salt thereof as disclosed in WO2019/047915.

The compound of Formula (I) or a pharmaceutically acceptable salt thereof disclosed in WO2019/047915 is illustrated as follows:

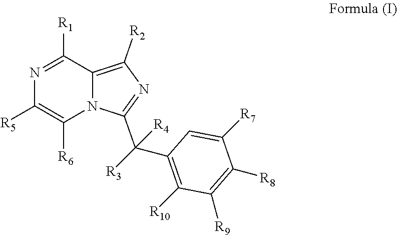

or a stereoisomer thereof, or a pharmaceutically acceptable salt thereof,
wherein:
R₁ is —NRₐR_{b}, wherein Rₐ and R_{b} are each independently hydrogen or C₁₋₆alkyl;
R₂ is hydrogen, F, Cl, Br, —C₁₋₆alkyl, —C₂₋₆alkenyl, —C₂₋₆alkynyl, cycloalkyl, heterocyclyl, aryl, heteroaryl, —CN, —NO₂, —OR₁₂, —SO₂R₁₂, —COR₁₂, —CO₂R₁₂, —CONR₁₂R₁₃, —C(═NR₁₂)NR₁₃R₁₄, —NR₁₂R₁₃, —NR₁₂COR₁₃, —NR₁₂CONR₁₃R₁₄, —NR₁₂CO₂R₁₃, —NR₁₂SONR₁₃R₁₄, —NR₁₂SO₂NR₁₃R₁₄, or —NR₁₂SO₂R₁₃; wherein said —C₁₋₆alkyl, —C₂₋₆alkenyl, —C₂₋₆alkynyl, cycloalkyl, heterocyclyl, aryl or heteroaryl are each independently optionally substituted with at least one substituent R₁₁ₐ;
R₃ and R₄, which may be the same or different, are each independently hydrogen, —C₁₋₆alkyl, cycloalkyl, heterocyclyl, aryl, or heteroaryl;
R₅ and R₆, which may be the same or different, are each independently hydrogen, halogen, —C₁₋₆alkyl, —C₂₋₆alkenyl, —C₂₋₆alkynyl, cycloalkyl, heterocyclyl, aryl, heteroaryl, —CN, —NO₂, —OR₁₂, —SO₂R₁₂, —COR₁₂, —CO₂R₁₂, —CONR₁₂R₁₃, —C(═NR₁₂)NR₁₃R₁₄, —NR₁₂R₁₃, —NR₁₂COR₁₃, —NR₁₂CONR₁₃R₁₄, —NR₁₂CO₂R₁₃, —NR₁₂SONR₁₃R₁₄, —NR₁₂SO₂NR₁₃R₁₄, or —NR₁₂SO₂R₁₃; wherein said —C₁₋₆alkyl,—C₂₋₆alkenyl, —C₂₋₆alkynyl, cycloalkyl, heterocyclyl, aryl or heteroaryl are each independently optionally substituted with at least one substituent R_{11b};
R₇, R₈ and R₁₀, which may be the same or different, are each independently hydrogen, halogen, —C₁₋₆alkyl, —C₂₋₆alkenyl, —C₂₋₆alkynyl, cycloalkyl, heterocyclyl, aryl, heteroaryl, —CN, —NO₂, —OR₁₂, —SO₂R₁₂, —COR₁₂, —CO₂R₁₂, —CONR₁₂R₁₃, —C(═NR₁₂)NR₁₃R₁₄, —NR₁₂R₁₃, —NR₁₂COR₁₃, —NR₁₂CONR₁₃R₁₄, —NR₁₂CO₂R₁₃, —NR₁₂SONR₁₃R₁₄, —NR₁₂SO₂NR₁₃R₁₄, or —NR₁₂SO₂R₁₃; wherein said —C₁₋₆alkyl, —C₂₋₆alkenyl, —C₂₋₆alkynyl, cycloalkyl, heterocyclyl, aryl or heteroaryl are each independently optionally substituted with at least one substituent R_{11c};
R₉ is —CN, —NO₂, —OR₁₂, —SO₂R₁₂, —SO₂NR₁₂R₁₃, —COR₁₂, —CO₂R₁₂, —CONR₁₂R₁₃, —C(═NR₁₂)NR₁₃R₁₄, —NR₁₂COR₁₃, —NR₁₂CONR₁₃R₁₄, —NR₁₂CO₂R₁₃, —NR₁₂SONR₁₃R₁₄, —NR₁₂SO₂NR₁₃R₁₄, or —NR₁₂SO₂R₁₃;
R₁₁ₐ, R_{11b}, and R_{11c}, which may be the same or different, are each independently hydrogen, halogen, —C₁₋₆alkyl, —C₂₋₆alkenyl, —C₂₋₆alkynyl, haloC₁₋₆alkyl, haloC₂₋₆alkenyl, haloC₂₋₆alkynyl, cycloalkyl, heterocyclyl, aryl, heteroaryl, —CN, —NO₂, oxo, —OR₁₂, —SO₂R₁₂, —COR₁₂, —CO₂R₁₂, —CONR₁₂R₁₃, —C(═NR₁₂)NR₁₃R₁₄, —NR₁₂R₁₃, —NR₁₂COR₁₃, —NR₁₂CONR₁₃R₁₄, —NR₁₂CO₂R₁₃, —NR₁₂SONR₁₃R₁₄, —NR₁₂SO₂NR₁₃R₁₄, or —NR₁₂SO₂R₁₃; and
R₁₂, R₁₃, and R₁₄, which may be the same or different, are each independently hydrogen, —C₁₋₆alkyl, —C₂₋₆alkenyl, —C₂₋₆alkynyl, cycloalkyl, heterocyclyl, aryl, or heteroaryl, wherein said C₁₋₆alkyl, —C₂₋₆alkenyl, —C₂₋₆alkynyl, cycloalkyl, heterocyclyl, aryl, or heteroaryl are each independently optionally substituted with at least one substituent R₁₅;

Alternatively, (R₁₂ and R₁₃), or (R₁₃ and R₁₄), or (R₁₂ and R₁₄), together with the atom(s) to which they are attached, form a 3- to 12-membered saturated, partially or fully unsaturated ring comprising 0, 1 or 2 additional heteroatoms independently selected from —NH, —O—, —S—, —SO— or —SO₂—, and said ring is optionally substituted with at least one substituent R₁₅;
R₁₅, at each of its occurrences, is independently hydrogen, halogen, —C₁₋₆alkyl, —C₂₋₆alkenyl, —C₂₋₆alkynyl, cycloalkyl, heterocyclyl, aryl, heteroaryl, —CN, —NO₂, oxo, —OR₁₆, —SO₂R₁₆, —COR₁₆, —CO₂R₁₆, —CONR₁₆R₁₇, —C(═NR₁₆)NR₁₇R₁₈, —NR₁₆R₁₇, —C₁₋₆alkyl-NR₁₆R₁₇, —NR₁₆COR₁₇, —NR₁₆CONR₁₇R₁₈, —NR₁₆CO₂R₁₇, —NR₁₆SONR₁₇R₁₈, —NR₁₆SO₂NR₁₇R₁₈, or —NR₁₆SO₂R₁₇, wherein said C₁₋₆alkyl, —C₂₋₆alkenyl, —C₂₋₆alkynyl, cycloalkyl, heterocyclyl, aryl, or heteroaryl are each independently optionally substituted with halogen, R₁₉, —OR₁₉, —COR₁₉, —SO₂R₁₉, or —CO₂R₁₉;
wherein each of R₁₆, R₁₇, or R₁₈ is independently hydrogen, —C₁₋₆alkyl, —C₂₋₆alkenyl, —C₂₋₆alkynyl, haloC₁₋₆alkyl, haloC₂₋₆alkenyl, haloC₂₋₆alkynyl, cycloalkyl, heterocyclyl, aryl, or heteroaryl; or
(R₁₆ and R₁₇), or (R₁₆ and R₁₈), or (R₁₇ and R₁₈), together with the atom(s) to which they are attached, form a 3- to 12-membered saturated, partially or fully unsaturated ring comprising 0, 1 or 2 additional heteroatoms independently selected from —NH, —O—, —S—, —SO— or —SO₂—, and said ring is optionally substituted with at least one substituent R₁₉; and
wherein R₁₉ is independently hydrogen, —C₁₋₆alkyl, —C₂₋₆alkenyl, —C₂₋₆alkynyl, haloC₁. 6alkyl, haloC₂₋₆alkenyl, haloC₂₋₆alkynyl, cycloalkyl, heterocyclyl, aryl, or heteroaryl, wherein said cycloalkyl, heterocyclyl, aryl, or heteroaryl are each optionally substituted with halogen, —C₁₋₆alkyl, —C₂₋₆alkenyl, —C₂₋₆alkynyl, haloC₁₋₆alkyl, haloC₂₋₆alkenyl, or haloC₂₋₆alkynyl; and wherein said —C₁₋₆alkyl, —C₂₋₆alkenyl, —C₂₋₆alkynyl, haloC₁₋₆alkyl, haloC₂₋₆alkenyl, or haloC₂₋₆alkynyl are each optionally substituted with cycloalkyl, heterocyclyl, aryl, or heteroaryl.

In the embodiments described above, the PI3Kδ inhibitor is (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide (Compound 1), or a pharmaceutically acceptable salt thereof.

The pharmaceutically acceptable salt of (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide) is a fumarate having the following formula

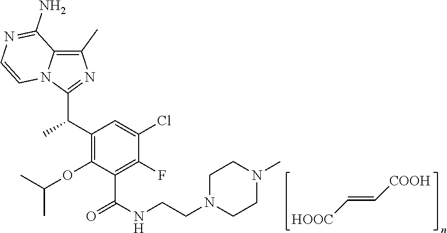

wherein n is a number from about 0.5 to about 2.0.

Preferably n is a number selected from the group consisting of 0.5±0.1, 1.0±0.2 and 1.5±0.2.

Preferably n is a number selected from 1.0±0.1, 1.1±0.1 and 1.5±0.1; preferably, n is 0.95˜1.05, 1.05˜1.15 or 1.45˜1.55; more preferably, n is 0.98˜1.02, 1.08˜1.12 or 1.48˜1.52; even more preferably, n is 1.0, 1.1 or 1.5.

The BTK inhibitor and the PI3Kδ inhibitor, are administered simultaneously, sequentially or intermittently.

A method for the treatment or delay of progression of cancer in a subject, comprising administering to the subject in need thereof a therapeutically effective amount of (S)-7-(1-acryloylpiperidin-4-yl)-2-(4-phenoxyphenyl)-4,5,6,7-tetra-hydropyrazolo[1,5-a]pyrimidine-3-carboxamide (BTK-1), or a pharmaceutically acceptable salt thereof, in combination with a therapeutically effective amount of a PI3Kδ inhibitor or a pharmaceutically acceptable salt thereof.

The embodiments described above, provide for a method wherein the PI3Kδ inhibitor is selected from the group consisting of: Idelalisib, Copanlisib, Duvelisib, Umbralisib, Leniolisib, Parsaclisib, AMG-319, ME-401, Tenalisib, Linperlisib, Seletalisib, Nemiralisib, KA-2237, SF-1126, HMPL-689, ACP-319, SHC-014748M, AZD-8154, PI3065 or a compound of Formula (I), or a pharmaceutically acceptable salt thereof.

The current disclosure provides for a method, wherein the PI3Kδ inhibitor is (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide (Compound 1) or a pharmaceutically acceptable salt thereof as disclosed in WO2019/047915.

In the embodiments described, provided is a method, wherein the cancer is hematologic cancer.

In the embodiment above wherein, the hematologic cancer is a leukemia, a lymphoma, a myeloma, a non-Hodgkin's lymphoma (NHL), a Hodgkin's lymphoma (HL), or a B-cell malignancy.

The embodiments above include a method wherein the B-cell malignancy is chronic lymphocytic leukemia (CLL), small lymphocytic lymphoma (SLL), follicular lymphoma (FL), mantle cell lymphoma (MCL), marginal zone lymphoma (MZL), Waldenstrom macroglobulinemia (WM), Hairy cell leukemia (HCL), Burkitt's-like leukemia (BL), B cell prolymphocytic leukemia (B-PLL), diffuse large B cell lymphoma (DLBCL), germinal center B-cell diffuse large B-cell lymphoma (GCB-DLBCL), non-germinal center B-cell diffuse large B-cell lymphoma (non-GCB DLBCL), DLBCL with undetermined subtype, primary central nervous system lymphoma (PCNSL), or secondary central nervous system lymphoma (SCNSL) of breast or testicular origin.

In the embodiments described above, wherein the method includes diffuse large B-cell lymphoma (DLBCL) is activated B-cell diffuse large B-cell lymphoma (ABC-DLBCL), GCB-DLBCL or non-GCB DLBCL.

The method, wherein the B-cell malignancy is a resistant B-cell malignancy.

The method, wherein the resistant B-cell malignancy is chronic lymphocytic leukemia (CLL), small lymphocytic lymphoma (SLL), follicular lymphoma (FL), mantle cell lymphoma (MCL), marginal zone lymphoma (MZL), Waldenstrom macroglobulinemia (WM), Hairy cell leukemia (HCL), Burkitt's-like leukemia (BL), B cell prolymphocytic leukemia (B-PLL), diffuse large B cell lymphoma (DLBCL), germinal center B-cell diffuse large B-cell lymphoma (GCB-DLBCL), non-germinal center B-cell diffuse large B-cell lymphoma (non-GCB DLBCL), DLBCL with undetermined subtype, primary central nervous system lymphoma (PCNSL), or secondary central nervous system lymphoma (SCNSL) of breast or testicular origin.

The embodiments described above, wherein the resistant B-cell malignancy is diffuse large B-cell lymphoma (DLBCL).

The method, wherein the resistant DLBCL is activated B-cell diffuse large B-cell lymphoma (ABC-DLBCL), GCB-DLBCL or non-GCB DLBCL.

In the embodiments described above, the cancer is selected from bladder cancer, breast cancer, colon cancer, gastroenterological cancer, kidney cancer, lung cancer (such as Non-small Cell Lung cancer), ovarian cancer, pancreatic cancer, prostate cancer, proximal or distal bile duct cancer, and melanoma.

The embodiments described above, provide for a method wherein the BTK inhibitor is administered at a dose of 50-600 mg QD or 20-320 mg BID. Preferably 50 mg, 100 mg, 150 mg, 200 mg, 250 mg, 300 mg, 350 mg, 400 mg, 450 mg, 500 mg, 550 mg or 600 mg QD or 20 mg, 40 mg, 60 mg, 80 mg, 100 mg, 120 mg, 140 mg, 160 mg, 180 mg, 200 mg, 220 mg, 240 mg, 260 mg, 280 mg, 300 mg or 320 mg BID.

The method, wherein the BTK inhibitor is administered at a dose of 320 mg QD or 160 mg BID.

The method, wherein the PI3Kδ inhibitor is administered at a dose of between 20 mg and 600 mg QD, such as 20-120 mg QD, 40-250 mg QD, 200-400 mg QD, 400-600 mg QD, 20 mg QD, 40 mg QD, 60 mg QD, 80 mg QD, 100 mg QD, 120 mg QD, 140 mg QD, 160 mg QD, 180 mg QD, 200 mg QD, 220 mg QD, 240 mg QD, 260 mg QD, 280 mg QD, 300 mg QD, 320 mg QD, 340 mg QD, 360 mg QD, 380 mg QD, 400 mg QD, 420 mg QD, 440 mg QD, 460 mg QD, 480 mg QD, 500 mg QD, 520 mg QD, 540 mg QD, 560 mg QD, or 580 mg QD. In another embodiment, the PI3Kδ inhibitor is administered at a dose of between 20 mg and 600 mg QD, such as 50 mg QD, 100 mg QD, 150 mg QD, 200 mg QD, 250 mg QD, 300 mg QD, 350 mg QD, 400 mg QD, 450 mg QD, 500 mg QD, 550 mg QD or 600 mg QD. In another embodiment, the PI3Kδ inhibitor is administered at a dose of between 20 mg and 320 mg BID, such as 20 mg BID, 40 mg BID, 60 mg BID, 80 mg BID, 100 mg BID, 120 mg BID, 140 mg BID, 160 mg BID, 180 mg BID, 200 mg BID, 220 mg BID, 240 mg BID, 260 mg BID, 280 mg BID, 300 mg BID or 320 mg BID. The dosage of the PI3Kδ inhibitor is between 5 mg to 80 mg per capsule, such as 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, or 80 mg per capsule.

In the embodiments described above, the PI3Kδ inhibitor is administered at a dose of 50 mg, 100 mg, 150 mg, 200 mg, 250 mg, 300 mg, 350 mg, 400 mg, 450 mg, 500 mg, 550 mg or 600 mg QD or 20 mg, 40 mg, 60 mg, 80 mg, 100 mg, 120 mg, 140 mg, 160 mg, 180 mg, 200 mg, 220 mg, 240 mg, 260 mg, 280 mg, 300 mg or 320 mg BID.

A pharmaceutical composition for use in the treatment or delay of progression of cancer, comprising administering to the subject in need thereof a therapeutically effective amount of (S)-7-(1-acryloylpiperidin-4-yl)-2-(4-phenoxyphenyl)-4,5,6,7-tetra-hydropyrazolo[1,5-a]pyrimidine-3-carboxamide (BTK-1), or a pharmaceutically acceptable salt thereof, in combination with a therapeutically effective amount of a PI3Kδ inhibitor or a pharmaceutically acceptable salt thereof.

In the embodiments described above, the PI3Kδ inhibitor is selected from the group consisting of: Idelalisib, Copanlisib, Duvelisib, Umbralisib, Leniolisib, Parsaclisib, AMG-319, ME-401, Tenalisib, Linperlisib, Seletalisib, Nemiralisib, KA-2237, SF-1126, HMPL-689, ACP-319, SHC-014748M, AZD-8154, PI3065 or a compound of Formula (I), or a pharmaceutically acceptable salt thereof as disclosed in WO2019/047915.

The embodiments described above, wherein the PI3Kδ inhibitor is (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide (Compound 1), or a pharmaceutically acceptable salt thereof.

A pharmaceutical combination for use in the treatment or delay of progression of cancer, comprising administering to the subject in need thereof a therapeutically effective amount of (S)-7-(1-acryloylpiperidin-4-yl)-2-(4-phenoxyphenyl)-4,5,6,7-tetra-hydropyrazolo[1,5-a]pyrimidine-3-carboxamide (BTK-1), or a pharmaceutically acceptable salt thereof, in combination with a therapeutically effective amount of a PI3Kδ inhibitor or a pharmaceutically acceptable salt thereof.

In the pharmaceutical combination, the PI3Kδ inhibitor is selected from the group consisting of: Idelalisib, Copanlisib, Duvelisib, Umbralisib, Leniolisib, Parsaclisib, AMG-319, ME-401, Tenalisib, Linperlisib, Seletalisib, Nemiralisib, KA-2237, SF-1126, HMPL-689, ACP-319, SHC-014748M, AZD-8154, PI3065, a compound of Formula (I), or a pharmaceutically acceptable salt thereof as disclosed in WO2019/047915.

In the pharmaceutical combination, the PI3Kδ inhibitor is (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide (Compound 1), or a pharmaceutically acceptable salt thereof.

Preferably, the pharmaceutically acceptable salt of Compound 1 is fumarate. The inventors have found that among different salts of Compound 1, fumarate salt of Compound 1 shows unpredictable high bioavailability, which makes the fumarate salt of Compound 1 suitable for pharmaceutical formulation.

Preferably, the PI3Kδ inhibitor is (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide fumarate (Compound 2).

In the embodiments described above, the PI3Kδ inhibitor is 6-[[4-(cyclopropylmethyl)-1-piperazinyl]methyl]-2-(5-fluoro-1H-indol-4-yl)-4-(4-morpholinyl)-thieno[3,2-d]pyrimidine (PI3065), or a pharmaceutically acceptable salt thereof.

The pharmaceutical combination for use, wherein the hematologic cancer is selected from leukemia, lymphoma, myeloma, non-Hodgkin's lymphoma (NHL), Hodgkin's lymphoma (HL), or B-cell malignancy.

The B-cell malignancy is chronic lymphocytic leukemia (CLL), small lymphocytic lymphoma (SLL), follicular lymphoma (FL), mantle cell lymphoma (MCL), marginal zone lymphoma (MZL), Waldenstrom macroglobulinemia (WM), Hairy cell leukemia (HCL), Burkitt's-like leukemia (BL), B cell prolymphocytic leukemia (B-PLL), diffuse large B cell lymphoma (DLBCL), germinal center B-cell diffuse large B-cell lymphoma (GCB-DLBCL), non-germinal center B-cell diffuse large B-cell lymphoma (non-GCB DLBCL), DLBCL with undetermined subtype, primary central nervous system lymphoma (PCNSL), secondary central nervous system lymphoma (SCNSL) of breast or testicular origin, or a combination of two or more thereof.

In the embodiments described above, the DLBCL is activated B-cell diffuse large B-cell lymphoma (ABC-DLBCL), GCB-DLBCL or non-GCB DLBCL.

In the embodiments described above, the B-cell malignancy is a resistant B-cell malignancy.

In the embodiments described above, the cancer is a sarcoma, or carcinoma.

In the embodiments described above, the cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma.

In the embodiments described above, the cancer is a resistant cancer.

### BRIEF DESCRIPTION OF THE DRAWINGS

FIG. 1 shows the ¹H-NMR spectrum for Compound 2 (freebase:fumarate=1:1).

FIG. 2 shows the combination of BTK1 and Compound 2 in an MCL xenograft model (JeKo-1 cells).

FIG. 3 shows the combination of BTK1 and Compound 2 in an MCL xenograft model (MINO cells).

FIG. 4 shows the combination of BTK1 and Compound 1 in an DLBCL xenograft model (TMD8 cells).

FIG. 5 shows the combination of BTK1 and Compound 2 in an DLBCL xenograft model (Farage cells).

### DETAILED DESCRIPTION OF THE DISCLOSURE

Abbreviations:

| | |
|---|---|
| ABC-DLBCL | Activated B-cell diffuse large B-cell lymphoma |
| B-PLL | B cell prolymphocytic leukemia |
| BTK | Bruton's Tyrosine Kinase |
| BTLA | B and T Lymphocyte Attenuator, CD272 |
| CLL | chronic lymphocytic leukemia |
| DLBCL | diffuse large B-cell lymphoma |
| DMEM | Dulbecco minimum essential medium |
| non-CLL/SLL | non-chronic lymphocytic leukemia/ |
| | small lymphocytic lymphoma |
| i.p. | Intraperitoneal or Intraperitoneally |
| NK | Natural killer |
| PBMC | Peripheral blood mononuclear cell |
| PBS | Phosphate Buffered Saline |
| PI3Kδ | phosphatidylinositol-3-kinase δ |
| p.o. | “by mouth” or “per os” |
| QD | Once daily |
| Q4D | Once every four days |
| QW | Once weekly |
| Q2W | Once every two weeks |
| Q3W | Once every three weeks |
| SLL | small lymphocytic lymphoma |
| mpk | mg/kg |

### Definitions

Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a,” “an,” and “the,” include their corresponding plural references unless the context clearly dictates otherwise.

The term “or” is used to mean, and is used interchangeably with, the term “and/of” unless the context clearly dictates otherwise.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” as used herein means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

It may be advantageous to separate reaction products from one another and/or from starting materials. The desired products of each step or series of steps is separated and/or purified (hereinafter separated) to the desired degree of homogeneity by the techniques common in the art. Typically, such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

“Diastereomers” refers to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents [Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302]. Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

“Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt can be prepared in situ during the final isolation and purification of the compounds disclosed herein, or separately by reacting the free base function with a suitable organic acid or by reacting the acidic group with a suitable base.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” includes salts of at least one compound of Formula (I), and salts of the stereoisomers of the compound of Formula (I), such as salts of enantiomers, and/or salts of diastereomers as disclosed in WO2019/047915.

The terms “administration,” “administering,” “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human.

The term “therapeutically acceptable amount” or “therapeutically effective dose” interchangeably refers to an amount sufficient to affect the desired result (i.e., a reduction in tumor size, inhibition of tumor growth, prevention of metastasis, inhibition or prevention of viral, bacterial, fungal or parasitic infection). In some aspects, a therapeutically acceptable amount does not induce or cause undesirable side effects. A therapeutically acceptable amount can be determined by first administering a low dose, and then incrementally increasing that dose until the desired effect is achieved. A “prophylactically effective dosage,” and a “therapeutically effective dosage,” of the molecules of the present disclosure can prevent the onset of, or result in a decrease in the severity of, respectively, disease symptoms, including symptoms associated polyoma viral infection.

The term “co-administer” refers to the simultaneous presence of two active agents in the blood of an individual. Active agents that are co-administered can be concurrently or sequentially delivered.

An “effective amount” refers to an amount of at least one compound and/or at least one stereoisomer thereof, and/or at least one pharmaceutically acceptable salt thereof effective to “treat” a disease or disorder in a subject, and that will elicit, to some significant extent, the biological or medical response of a tissue, system, animal or human that is being sought, such as when administered, is sufficient to prevent the development of, or alleviate to some extent, one or more of the symptoms of the condition or disorder being treated. The therapeutically effective amount will vary depending on the compound, the disease and its severity and the age, weight, etc., of the mammal to be treated.

The terms “cancer” or “tumor” herein mean or describe the physiological condition involving abnormal cell growth with the potential to invade or spread to other parts of the body.

As used herein, the term “resistant,” “resistant cancer” or “refractory” refers to a condition wherein the cancer demonstrates reduced sensitivity to a therapeutic. For example, in a resistant cancer, fewer cancer cells are eliminated by the concentration of a therapeutic that is used to eliminate cancer cells in a sensitive cancer of the same type. A cancer can be resistant at the beginning of a therapeutic treatment or it can become resistant during treatment. Resistance can be due to several mechanisms such as but not limited to; alterations in drug-targets, decreased drug accumulation, alteration of intracellular drug distribution, reduced drug-target interaction, increased detoxification response, cell-cycle deregulation, increased damaged-DNA repair, and reduced apoptotic response. Several of said mechanisms can occur simultaneously and/or can interact with each other.

The term “solid tumor” refers to a tumor other than leukemia or lymphoma (ie, blood cancer) that forms a solid mass of cancer cells. As used herein, the term “advanced solid tumor” refers to a malignant tumor that is metastatic or locally progressing and cannot be operated on.

The term “disease” refers to any disease, discomfort, illness, symptoms or indications, and can be substituted with the term “disorder” or “condition.”

The term “pharmaceutical combination” as used herein refers to either a fixed combination in one dosage unit form, or non-fixed combination or a kit of parts for the combined administration where two or more therapeutic agents can be administered independently at the same time or separately within time intervals, especially where these time intervals allow that the combination partners show a cooperative, e.g., synergistic effect.

The term “combination therapy” refers to the administration of two or more therapeutic agents to treat cancer or a consequence of cancer as described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids can be reconstituted or diluted to a desired dose prior to administration. In addition, such administration also encompasses use of each type of therapeutic agent in a sequential manner, either at approximately the same time or at different times. In either case, the treatment regimen will provide beneficial effects of the therapeutic combination in treating the conditions or disorders described herein.

Unless specifically defined elsewhere in this document, all other technical and scientific terms used herein have the meaning commonly understood by one of ordinary skill in the art to which this invention belongs.

The combination therapy can provide “synergy” and prove “synergistic,” i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

BTK inhibitors

The BTK inhibitor disclosed herein, (S)-7-(1-acryloylpiperidin-4-yl)-2-(4-phenoxyphenyl)-4,5,6,7-tetra-hydropyrazolo[1,5-a]pyrimidine-3-carboxamide (BTK-1), can be synthesized by synthetic routes disclosed in WO2014/173289, the entire disclosure of which is expressly incorporated herein by reference.

### PI3Kδ Inhibitors

The “PI3Kδ inhibitor” includes but not limited to: Idelalisib, Copanlisib, Duvelisib, Umbralisib, Leniolisib, Parsaclisib, AMG-319, ME-401, Tenalisib, Linperlisib, Seletalisib, Nemiralisib, KA-2237, SF-1126, HMPL-689, ACP-319, SHC-014748M, AZD-8154, 6-[[4-(cyclopropylmethyl)-1-piperazinyl]methyl]-2-(5-fluoro-1H-indol-4-yl)-4-(4-morpholinyl)-thieno[3,2-d]pyrimidine (PI3065), a compound of Formula (I), or a pharmaceutically acceptable salt thereof as disclosed in WO2019/047915.

As disclosed herein, the PI3Kδ inhibitor is a compound of Formula (I),

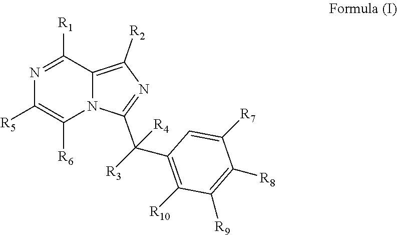

or a stereoisomer thereof, or a pharmaceutically acceptable salt thereof,
wherein:
R₁ is —NRₐR_{b}, wherein Rₐ and R_{b} are each independently hydrogen or C₁₋₆alkyl;
R₂ is hydrogen, F, Cl, Br, —C₁₋₆alkyl, —C₂₋₆alkenyl, —C₂₋₆alkynyl, cycloalkyl, heterocyclyl, aryl, heteroaryl, —CN, —NO₂, —OR₁₂, —SO₂R₁₂, —COR₁₂, —CO₂R₁₂, —CONR₁₂R₁₃, —C(═NR₁₂)NR₁₃R₁₄, —NR₁₂R₁₃, —NR₁₂COR₁₃, —NR₁₂CONR₁₃R₁₄, —NR₁₂CO₂R₁₃, —NR₁₂SONR₁₃R₁₄, —NR₁₂SO₂NR₁₃R₁₄, or —NR₁₂SO₂R₁₃; wherein said —C₁₋₆alkyl, —C₂₋₆alkenyl, —C₂₋₆alkynyl, cycloalkyl, heterocyclyl, aryl or heteroaryl are each independently optionally substituted with at least one substituent R₁₁ₐ;
R₃ and R₄, which may be the same or different, are each independently hydrogen, —C₁₋₆ alkyl, cycloalkyl, heterocyclyl, aryl, or heteroaryl;
R₅ and R₆, which may be the same or different, are each independently hydrogen, halogen, —C₁₋₆alkyl, —C₂₋₆alkenyl, —C₂₋₆alkynyl, cycloalkyl, heterocyclyl, aryl, heteroaryl, —CN, —NO₂, —OR₁₂, —SO₂R₁₂, —COR₁₂, —CO₂R₁₂, —CONR₁₂R₁₃, —C(═NR₁₂)NR₁₃R₁₄, —NR₁₂R₁₃, —NR₁₂COR₁₃, —NR₁₂CONR₁₃R₁₄, —NR₁₂CO₂R₁₃, —NR₁₂SONR₁₃R₁₄, —NR₁₂SO₂NR₁₃R₁₄, or —NR₁₂SO₂R₁₃; wherein said —C₁₋₆alkyl, —C₂₋₆alkenyl, —C₂₋₆alkynyl, cycloalkyl, heterocyclyl, aryl or heteroaryl are each independently optionally substituted with at least one substituent R_{11b};
R₇, R₈ and R₁₀, which may be the same or different, are each independently hydrogen, halogen, —C₁₋₆alkyl, —C₂₋₆alkenyl, —C₂₋₆alkynyl, cycloalkyl, heterocyclyl, aryl, heteroaryl, —CN, —NO₂, —OR₁₂, —SO₂R₁₂, —COR₁₂, —CO₂R₁₂, —CONR₁₂R₁₃, —C(═NR₁₂)NR₁₃R₁₄, —NR₁₂R₁₃, —NR₁₂COR₁₃, —NR₁₂CONR₁₃R₁₄, —NR₁₂CO₂R₁₃, —NR₁₂SONR₁₃R₁₄, —NR₁₂SO₂NR₁₃R₁₄, or —NR₁₂SO₂R₁₃; wherein said —C₁₋₆alkyl, —C₂₋₆alkenyl, —C₂₋₆alkynyl, cycloalkyl, heterocyclyl, aryl or heteroaryl are each independently optionally substituted with at least one substituent R_{11c};
R₉ is —CN, —NO₂, —OR₁₂, —SO₂R₁₂, —SO₂NR₁₂R₁₃, —COR₁₂, —CO₂R₁₂, —CONR₁₂R₁₃, —C(═NR₁₂)NR₁₃R₁₄, —NR₁₂COR₁₃, —NR₁₂CONR₁₃R₁₄, —NR₁₂CO₂R₁₃, —NR₁₂SONR₁₃R₁₄, —NR₁₂SO₂NR₁₃R₁₄, or —NR₁₂SO₂R₁₃;
R₁₁ₐ, R_{11b}, and R_{11c}, which may be the same or different, are each independently hydrogen, halogen, —C₁₋₆alkyl, —C₂₋₆alkenyl, —C₂₋₆alkynyl, haloC₁₋₆alkyl, haloC₂₋₆alkenyl, haloC₂₋₆alkynyl, cycloalkyl, heterocyclyl, aryl, heteroaryl, —CN, —NO₂, oxo, —OR₁₂, —SO₂R₁₂, —COR₁₂, —CO₂R₁₂, —CONR₁₂R₁₃, —C(═NR₁₂)NR₁₃R₁₄, —NR₁₂R₁₃, —NR₁₂COR₁₃, —NR₁₂CONR₁₃R₁₄, —NR₁₂CO₂R₁₃, —NR₁₂SONR₁₃R₁₄, —NR₁₂SO₂NR₁₃R₁₄, or —NR₁₂SO₂R₁₃; and
R₁₂, R₁₃, and R₁₄, which may be the same or different, are each independently hydrogen, —C₁₋₆alkyl, —C₂₋₆alkenyl, —C₂₋₆alkynyl, cycloalkyl, heterocyclyl, aryl, or heteroaryl, wherein said C₁₋₆alkyl, —C₂₋₆alkenyl, —C₂₋₆alkynyl, cycloalkyl, heterocyclyl, aryl, or heteroaryl are each independently optionally substituted with at least one substituent R₁₅;

Alternatively, (R₁₂ and R₁₃), or (R₁₃ and R₁₄), or (R₁₂ and R₁₄), together with the atom(s) to which they are attached, form a 3- to 12-membered saturated, partially or fully unsaturated ring comprising 0, 1 or 2 additional heteroatoms independently selected from —NH, —O—, —S—, —SO— or —SO₂—, and said ring is optionally substituted with at least one substituent R₁₅;
R₁₅, at each of its occurrences, is independently hydrogen, halogen, —C₁₋₆alkyl, —C₂₋₆alkenyl, —C₂₋₆alkynyl, cycloalkyl, heterocyclyl, aryl, heteroaryl, —CN, —NO₂, oxo, —OR₁₆, —SO₂R₁₆, —COR₁₆, —CO₂R₁₆, —CONR₁₆R₁₇, —C(═NR₁₆)NR₁₇R₁₈, —NR₁₆R₁₇, —C₁₋₆alkyl-NR₁₆R₁₇, —NR₁₆COR₁₇, —NR₁₆CONR₁₇R₁₈, —NR₁₆CO₂R₁₇, —NR₁₆SONR₁₇R₁₈, —NR₁₆SO₂NR₁₇R₁₈, or —NR₁₆SO₂R₁₇, wherein said C₁₋₆alkyl, —C₂₋₆alkenyl, —C₂₋₆alkynyl, cycloalkyl, heterocyclyl, aryl, or heteroaryl are each independently optionally substituted with halogen, R₁₉, —OR₁₉, —COR₁₉, —SO₂R₁₉, or —CO₂R₁₉;
wherein each of R₁₆, R₁₇, or R₁₈ is independently hydrogen, —C₁₋₆alkyl, —C₂₋₆alkenyl, —C₂₋₆alkynyl, haloC₁₋₆alkyl, haloC₂₋₆alkenyl, haloC₂₋₆alkynyl, cycloalkyl, heterocyclyl, aryl, or heteroaryl; or
(R₁₆ and R₁₇), or (R₁₆ and R₁₈), or (R₁₇ and R₁₈), together with the atom(s) to which they are attached, form a 3- to 12-membered saturated, partially or fully unsaturated ring comprising 0, 1 or 2 additional heteroatoms independently selected from —NH, —O—, —S—, —SO— or —SO₂—, and said ring is optionally substituted with at least one substituent R₁₉; and
wherein R₁₉ is independently hydrogen, —C₁₋₆alkyl, —C₂₋₆alkenyl, —C₂₋₆alkynyl, haloC₁₋₆alkyl, haloC₂₋₆alkenyl, haloC₂₋₆alkynyl, cycloalkyl, heterocyclyl, aryl, or heteroaryl, wherein said cycloalkyl, heterocyclyl, aryl, or heteroaryl are each optionally substituted with halogen, —C₁₋₆alkyl, —C₂₋₆alkenyl, —C₂₋₆alkynyl, haloC₁₋₆alkyl, haloC₂₋₆alkenyl, or haloC₂₋₆alkynyl; and wherein said —C₁₋₆alkyl, —C₂₋₆alkenyl, —C₂₋₆alkynyl, haloC₁₋₆alkyl, haloC₂₋₆alkenyl, or haloC₂₋₆ alkynyl are each optionally substituted with cycloalkyl, heterocyclyl, aryl, or heteroaryl.

In some embodiments, wherein the carbon atom to which R₃ and R₄ are attached is in (S)-configuration when R₃ and R₄ are different.

As disclosed herein, the PI3Kδ inhibitor is (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide (Compound 1).

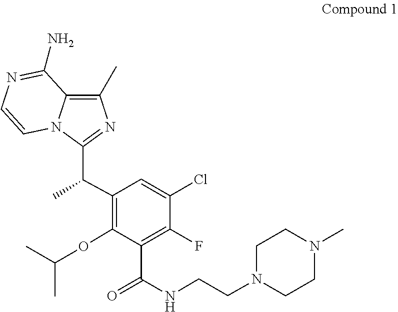 [SMILES: Cc1nc([C@@H](C)c2cc(Cl)c(F)c(C(=O)NCCN3CCN(C)CC3)c2OC(C)C)n2ccnc(N)c12]

The PI3Kδ inhibitor disclosed herein can be synthesized by synthetic routes disclosed in WO2019047915, the entire disclosure of which is expressly incorporated herein by reference.

As disclosed herein, the PI3Kδ inhibitor is (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide fumarate.

The pharmaceutically acceptable salt of (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide) is fumarate having the following formula

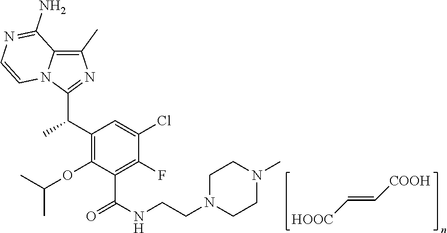

wherein n is a number from about 0.5 to about 2.0.

Preferably n is a number selected from the group consisting of 0.5±0.1, 1.0±0.2 and 1.5±0.2.

Preferably n is a number selected from 1.0±0.1, 1.1±0.1 and 1.5±0.1; preferably, n is 0.95˜1.05, 1.05˜1.15 or 1.45˜1.55; more preferably, n is 0.98˜1.02, 1.08˜1.12 or 1.48˜1.52; even more preferably, n is 1.0, 1.1 or 1.5.

### Combination Therapy

The combination therapy can be administered as a simultaneous or separate or sequential regimen. When administered sequentially, the combination can be administered in two or more administrations. The combined administration includes co-administration, using the separate formulation, and consecutive administration in either order, wherein preferably there is a time period while both (or all) active agents simultaneously exert their biological activities.

Suitable dosages for any of the above co-administered agents are those presently used and can be adjusted due to the combined action of the BTK inhibitor and the PI3Kδ inhibitor, such as to increase the therapeutic index or mitigate toxicity or other side-effects or consequences.

In another embodiment, the amounts of the BTK inhibitor and the PI3Kδ inhibitor disclosed herein and the relative timings of administration can be determined by the individual needs of the patient to be treated, administration route, the severity of disease or illness, dosing schedule, as well as evaluation and judgment of the prescribing physician.

Methods for co-administration or treatment with an additional therapeutic agent, e.g., an immunosuppressant, a cytokine, steroid, chemotherapeutic agent, antibiotic, or radiation, are known in the art (see, e.g., Hardman et al., (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). An effective amount of the additional therapeutic agent can decrease the symptoms by at least 10%; by at least 20%; at least about 30%; at least 40%, or at least 50%.

The present disclosure provides for the combination therapies to also be cyclically administered. Cycling therapy involves the administration of a first therapy (e.g., a first compound or therapeutic agent) for a period of time, followed by the administration of a second therapy (e.g., a second compound or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve the efficacy of the therapies.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents can be administered to a subject by the same or different routes of administration.

The BTK inhibitor and the PI3Kδ inhibitor of the present disclosure can also be administered via one or more routes of administration using one or more of a variety of methods known in the art. As will be appreciated by the skilled artisan, the route and/or mode of administration will vary depending upon the desired results. Selected routes of administration for the composition or combination include intravenous, intramuscular, for example by injection or infusion. Alternatively, the combination or each individual agent of the present disclosure can be administered via a non-parenteral route, for example, orally.

In one embodiment, a BTK inhibitor and a PI3Kδ inhibitor disclosed herein can be administered in different routes. In one embodiment, a BTK inhibitor is administered orally, and a PI3Kδ inhibitor is also administered orally. In another embodiment, a BTK inhibitor is administered orally, and a PI3Kδ inhibitor is administered parenterally.

The BTK inhibitor can be administered once per month, twice per month, once a week, twice a week, once a day, two times per day, three times per day, four times per day, or five times per day. The BTK inhibitor can be administered at a dosage from 50 mg to 600 mg, such as about 50 mg, 100 mg, 150 mg, 200 mg, 250 mg, 300 mg, 350 mg, 400 mg, 450 mg, 500 mg, 550 mg or 600 mg QD or from 20 mg to 320 mg, such as 20 mg, 40 mg, 60 mg, 80 mg, 100 mg, 120 mg, 140 mg, 160 mg, 180 mg, 200 mg, 220 mg, 240 mg, 260 mg, 280 mg, 300 mg or 320 mg BID. In one embodiment, the BTK inhibitor is administered at a dose of 320 mg QD or 160 mg BID.

The PI3Kδ inhibitor can be administered once per month, twice per month, once a week, twice a week, once a day, two times per day, three times per day, four times per day, or five times per day. The PI3Kδ inhibitor can be administered at a dosage from about 20 mg/day, 30 mg/day, 40 mg/day, 50 mg/day, 60 mg/day, 70 mg/day, 80 mg/day, 100 mg/day, 200 mg/day, 300 mg/day, 400 mg/day, 500 mg/day, 600 mg/day, 700 mg/day, 800 mg/day, 900 mg/day to 1000 mg/day.

The therapeutic agents of the disclosure can be administered to a subject concurrently. The term “concurrently” is not limited to the administration of each compound or therapeutic agent at exactly the same time, but rather it is meant that a pharmaceutical composition comprising one therapeutic agent is administered to a subject in a sequence and within a time interval such that one therapeutic can act together with the other therapeutic to provide an increased benefit than if they were administered otherwise. For example, each therapy can be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route. In various aspects, the therapeutic agents are administered to a subject less than 15 minutes, less than 30 minutes, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, 24 hours apart, 48 hours apart, 72 hours apart, or 1 week apart. In other aspects, the individual therapeutic agents are administered within the same patient visit.

### EXAMPLES

### Preparation of Compound 1

### (S)-3-(1-(8-amino-1-methylimidazo[1,5-a]pyrazin-3-yl)ethyl)-5-chloro-6-fluoro-2-isopropoxy-N-(2-(4-methylpiperazi-1-yl)ethyl)benzamide

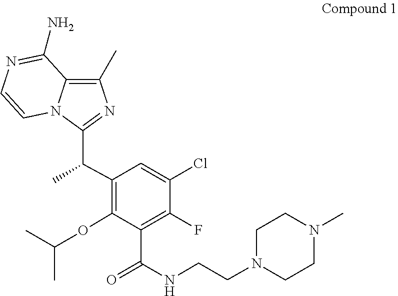 [SMILES: Cc1nc([C@@H](C)c2cc(Cl)c(F)c(C(=O)NCCN3CCN(C)CC3)c2OC(C)C)n2ccnc(N)c12]

Compound 1 (93 mg, 72.1%) was prepared from 2-(4-methylpiperazin-1-yl)ethan-1-amine by using the steps below.

### Step 1: 1-(5-chloro-4-fluoro-2-hydroxyphenyl) ethan-1-one

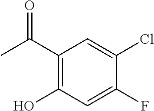

To a 2 L three-necked flask equipped with a magnetic stirrer was added 4-chloro-3-fluorophenol (160 g, 1.1 mol) and acetyl chloride (129 g, 1.6 mmol). The mixture was stirred for 1 h. Then aluminum chloride (219 g, 1.6 mmol) was added into the mixture in portions. The mixture was heated to 160° C. and kept at 160° C. for 2 hrs. The mixture was cooled and diluted with HCl (2 M, 500 mL). The resulting hot liquid was cooled and extracted with EtOAc (500 mL×3). The combined organic phase was washed with water (500 mL), and brine (500 mL), dried over anhydrous sodium sulfate, filtered and concentrated to give the product (200 g, crude) as a yellow solid. ¹H NMR (400 MHz, CDCl3) δ 12.48-12.41 (m, 1H), 7.78 (d, J=8.1 Hz, 1H), 6.77 (d, J=10.3 Hz, 1H), 2.61 (s, 3H).

### Step 2: 1-(3-bromo-5-chloro-4-fluoro-2-hydroxyphenyl) ethan-1-one

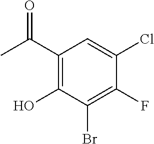

To a solution of 1-(5-chloro-4-fluoro-2-hydroxyphenyl) ethan-1-one (110 g, 583 mmol) in DMF (1 L) was added NBS (114 g, 640 mmol) in portions. The mixture was stirred at room temperature for 1 h. The mixture was diluted with water (3 L), extracted with EtOAc (1 L×3). The combined organic phase was washed with brine (1 L×3), dried over anhydrous sodium sulfate, filtered and concentrated to give the product (150 g, crude) as a yellow solid. ¹H NMR (400 MHz, CDCl3) δ 13.21 (d, brs, 1H), 7.80 (d, J=7.8 Hz, 1H), 2.66 (s, 3H).

### Step 3:1-(3-bromo-5-chloro-4-fluoro-2-isopropoxyphenyl) ethan-1-one

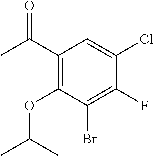 [SMILES: CC(=O)c1cc(Cl)c(F)c(Br)c1OC(C)C]

To a solution of 1-(3-bromo-5-chloro-4-fluoro-2-hydroxyphenyl) ethan-1-one (150 g, 560 mmol) and 2-iodopropane (143 g, 841 mmol) in DMF (1 L) was added NaHCO₃ (71 g, 845 mmol). The mixture was stirred at 60° C. for overnight. The mixture was cooled and diluted with water (3 L), extracted with EtOAc (1 L×3). The combined organic phase was washed with brine (1 L×3), dried over anhydrous sodium sulfate, filtered and concentrated. The residue was purified by silica gel column chromatography (elution with hexane/ethyl acetate=50/1) to give the product (140 g, 80%) as a yellow oil. ¹H NMR (400 MHz, CDCl3) δ 7.57 (d, J=8.2 Hz, 1H), 4.45-4.39 (m, 1H), 2.61 (s, 3H), 1.31 (t, J=6.7 Hz, 6H).

### Step 4:2-(3-bromo-5-chloro-4-fluoro-2-isopropoxyphenyl) propanenitrile

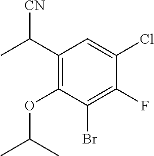 [SMILES: CC(C)Oc1c(C(C)C#N)cc(Cl)c(F)c1Br]

1-(3-bromo-5-chloro-4-fluoro-2-isopropoxyphenyl) ethan-1-one (165 g, 533 mmol) in DME (420 mL) was added TOSMIC (156 g, 799 mmol), the solution was stirred at 0° C. A solution of t-BuOK (119.6 g, 1066 mmol) in t-BuOH (840 mL) was added to the above solution by dropwise under N₂ and maintained the temperature below 10° C., the resulting solution was stirred at room temperature for overnight. After completion, the reaction mixture was washed with water (1 L) and extracted with ethyl acetate (500 mL×3), dried over MgSO4, filtered and evaporated in vacuo, The residue was purified by column chromatography (PE/EA=20:1˜10:1) to give the product (118 g, 69.2%) as yellow solid. ¹H NMR (400 MHz, CDCl3) δ 7.51 (d, J=7.8 Hz, 1H), 4.69 (dt, J=12.3, 6.2 Hz, 1H), 4.31 (q, J=7.2 Hz, 1H), 1.56 (d, J=7.2 Hz, 3H), 1.44 (d, J=6.2 Hz, 3H), 1.30 (d, J=6.2 Hz, 3H).

### Step 5: 2-(3-bromo-5-chloro-4-fluoro-2-isopropoxyphenyl) propanoic acid

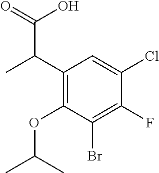 [SMILES: CC(C)Oc1c(C(C)C(=O)O)cc(Cl)c(F)c1Br]

2-(3-bromo-5-chloro-4-fluoro-2 isopropoxyphenyl) propanenitrile (118 g, 369 mmol) in EtOH (307 mL) was added a.q. NaOH (6 N, 307 mL), the resulting solution was stirred at 100° C. for overnight. After completion, the reaction was cooled to room temperature, adjusted pH to 3˜4 by addition of 1N HCl, extracted with ethyl acetate (500 mL×3), the combined ethyl acetate phrase was dried over MgSO4, filtered and evaporated to give the crude product (122 g, 97.4%) as yellow oil which was used in the next step without further purification. LC-MS (M−H)⁺=336.9.

### Step 6: (S)-2-(3-bromo-5-chloro-4-fluoro-2-isopropoxyphenyl) propanoic acid

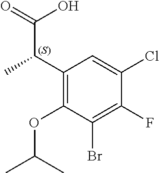

2-(3-bromo-5-chloro-4-fluoro-2 isopropoxyphenyl) propanoic acid (122 g, 359 mmol) and (1R,2S)-1-amino-2,3-dihydro-1H-inden-2-ol (54 g, 359 mmol) in i-PrOH (500 mL) was stirred at 100° C. for 1 h, cooled to room temperature, concentrated to afford crude salt, which was slurried in PE/EA=10:1 (500 mL) for 1˜2 h, undissolved solid was collected and refluxed in PE/EA/i-PrOH=20:2:1 (230 mL) for another 1 h, the solid was collected by filtration and dried in vacuo to give the chiral salt which was neutralized by addition of aq. HCl (1N) to pH to 2˜3, extracted with ethyl acetate (200 mL×3), dried over MgSO4, concentrated to afford the product as yellow oil (44.2 g, 36.2%). ¹H NMR (400 MHz, DMSO-d6), δ 12.59 (s, 1H), 7.52 (d, J=8.4 Hz, 1H), 4.55 (dt, J=12.3, 6.1 Hz, 1H), 4.04 (q, J=7.0, 1H), 1.38 (d, J=7.3 Hz, 3H), 1.34-1.26 (m, 6H). LC-MS (M−H)+=336.9. RetTime in chiral-HPLC: 2.61 min. The absolute (S) configuration of chiral center was confirmed by x-ray analysis of single crystal.

### Step 7: (2S)-2-(3-bromo-5-chloro-4-fluoro-2-isopropoxyphenyl)-N-(1-(3-chloropyrazin-2-yl) ethyl) propanamide

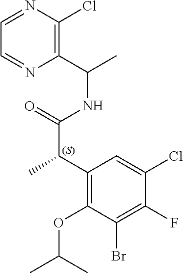

(S)-2-(3-bromo-5-chloro-4-fluoro-2-isopropoxyphenyl) propanoic acid (52 g, 153 mmol), 1-(3-chloropyrazin-2-yl) ethan-1-amine hydrochloride (29.7 g, 153 mmol), EDCI (43.9 g, 229.7 mmol), HOBT (31 g, 229.7 mmol) and Et₃N (49.5 g, 489.6 mmol) in DCM (500 mL) was stirred at room temperature overnight under N₂. After completion, the reaction solution was washed with H2O (500 mL), extracted with DCM (500 mL×3), combined DCM phase was dried over MgSO4, concentrated and purified by column chromatography (PE/EA=10:1˜5:1) to give the product (69 g, 94%) as yellow oil. LC-MS (M+H)+=479.6.

### Step 8: (S)-3-(1-(3-bromo-5-chloro-4-fluoro-2-isopropoxyphenyl) ethyl)-8-chloro-1-methylimidazo [1,5-a] pyrazine

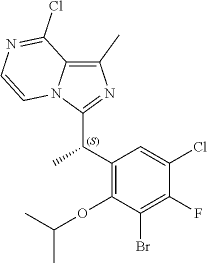

(2S)-2-(3-bromo-5-chloro-4-fluoro-2-isopropoxyphenyl)-N-(1-(3-chloropyrazin-2-yl) ethyl) propanamide (69 g, 144 mmol) in DCM (1 L) was added Tf₂O (89.4 g, 317 mmol) by dropwise at 0° C., then pyridine (28.5 g, 360 mmol) was added by dropwise at 0° C., TLC showed the reaction was completed, H₂O (500 mL) was added, extracted with DCM (500 mL×3), combined DCM phase was dried over MgSO₄, concentrated to afford crude product which was slurried in i-PrOH (60 mL) for 1˜2 h, filtrated to give pure product as white solid (55 g, 83.4%). LC-MS (M+H)⁺=461.9.

### Step 9: (S)-3-(1-(3-bromo-5-chloro-4-fluoro-2-isopropoxyphenyl) ethyl)-1-methylimidazo [1,5-a] pyrazin-8-amine

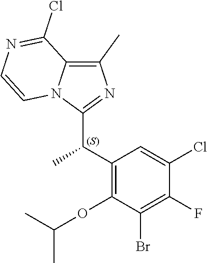

To a pressure tank equipped with a magnetic stirrer were added (S)-3-(1-(3-bromo-5-chloro-4-fluoro-2-isopropoxyphenyl) ethyl)-8-chloro-1-methylimidazo [1,5-a] pyrazine (45 g, 97.6 mmol) and NH₃ in i-PrOH (w/w 30%, 300 mL, excess). Then the mixture was stirred at 90° C. for two days. The mixture was cooled and diluted with DCM (500 mL), washed with water (100 mL×3), brine (100 mL), dried over Na₂SO₄, filtered and concentrated to give the product (41 g, 95%) as a yellow solid which was used in the next step without further purification. ¹H NMR (400 MHz, CDCl3) δ 7.27 (d, J=7.6 Hz, 1H), 7.15 (d, J=5.1 Hz, 1H), 6.88 (d, J=5.0 Hz, 1H), 4.78-4.69 (m, 2H), 2.72 (s, 3H), 1.80 (d, J=7.2 Hz, 3H), 1.49 (d, J=6.2 Hz, 3H), 1.39 (d, J=6.2 Hz, 3H). LC-MS (M+H)⁺=441.0, 443.0.

### Step 10: (S)-6-(1-(8-amino-1-methylimidazo [1,5-a] pyrazin-3-yl)ethyl)-2-bromo-4-chloro-3-fluorophenol

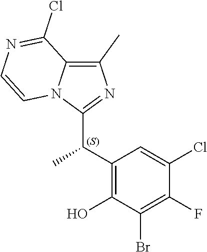

To a mixture of (S)-3-(1-(3-bromo-5-chloro-4-fluoro-2-isopropoxyphenyl) ethyl)-1-methylimidazo [1,5-a] pyrazin-8-amine (41 g, 92.8 mmol) in DCM (500 mL) was added BBr₃ (70 g, 279 mmol) by dropwise at 0° C. Then the mixture was stirred at room temperature overnight. The mixture was cooled to 0° C., and then quenched with MeOH (400 mL). The mixture was concentrated, the residue was diluted with a mixture of DCM (500 mL) and i-PrOH (100 mL). Then the mixture was washed with saturated NaHCO₃ solution (100 mL×2). The organic layers were separated, washed with brine, dried over Na₂SO₄, filtered and concentrated to give the product (38 g, 100%) as a yellow solid which was used for the next step without further purification. ¹H NMR (400 MHz, CDCl3) δ 7.18 (d, J=5.2 Hz, 1H), 7.12 (d, J=7.9 Hz, 1H), 7.02 (d, J=5.1 Hz, 1H), 4.28 (q, J=7.3 Hz, 1H), 4.08-398 (m, 1H), 2.72 (s, 3H), 1.70 (d, J=7.3 Hz, 3H), 1.21 (d, J=6.1 Hz, 6H). LC-MS (M+H)+=399.0, 401.0.

### Step 11: ethyl (S)-3-(1-(8-amino-1-methylimidazo[1,5-a]pyrazin-3-yl)ethyl)-5-chloro-6-fluoro-2-hydroxybenzoate

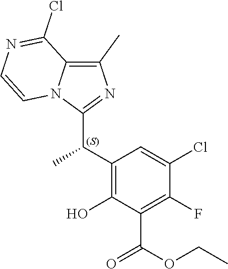

To a mixture of (S)-6-(1-(8-amino-1-methylimidazo [1,5-a] pyrazin-3-yl) ethyl)-2-bromo-4-chloro-3-fluorophenol (38 g, 32.5 mmol) in EtOH (1000 mL) were added Pd (dppf) Cl₂ (3.5 g, 4.8 mmol) and NaOAc (11.7 g, 143 mmol). The mixture was degassed and refilled with CO (1 atm). The mixture was stirred at 70° C. for overnight. The mixture was cooled down and concentrated in vacuo. The residue was diluted with water (200 mL), extracted with EtOAc (200 mL×3). The combined organic phase was washed with brine, dried over Na₂SO₄, filtered and concentrated. The residue was purified by column chromatography (DCM/MeOH from DCM 100% to 20/1) to give the product (32 g, 82%) as a yellow solid. ¹H NMR (400 MHz, CDCl₃) δ 7.28-7.24 (m, 1H), 7.07 (d, J=5.1 Hz, 1H), 6.85 (d, J=5.1 Hz, 1H), 5.30 (s, 1H), 4.81 (q, J=7.1 Hz, 1H), 4.48 (q, J=7.1 Hz, 2H), 2.75 (s, 3H), 1.74 (d, J=7.1 Hz, 3H), 1.43 (t, J=7.1 Hz, 3H). LC-MS (M+H)⁺=393.1.

### Step 12: ethyl (S)-3-(1-(8-amino-1-methylimidazo [1,5-a]pyrazin-3-yl)ethyl)-5-chloro-6-fluoro-2-isopropoxybenzoate

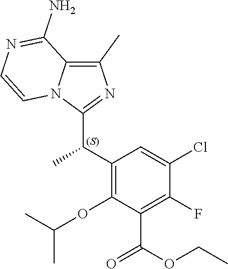

ethyl (S)-3-(1-(8-amino-1-methylimidazo [1,5-a] pyrazin-3-yl) ethyl)-5-chloro-6-fluoro-2-hydroxybenzoate (32 g, 81.5 mmol), i-PrOH (24.4 g, 406.7 mmol), PPh₃ (49.1 g, 187.5 mmol) in toluene (400 mL) was added di-tert-butyl (E)-diazene-1,2-dicarboxylate (43.2 g, 187.5 mmol) at room temperature. The resulting solution was stirred at 60° C. for 3 hrs under N₂. After completion, the reaction mixture was concentrated in vacuo, washed with H₂O (500 mL), extracted with EtOAc (500 mL×3), combined EtOAc phase was dried over MgSO₄, purified by column chromatography (PE/EA=20:1) to give the product (25.4 g, 71.8%) as yellow solid. LC-MS (M+H)+=435.1.

### Step 13: (S)-3-(1-(8-amino-1-methylimidazo[1,5-a]pyrazin-3-yl) ethyl)-5-chloro-6-fluoro-2-isopropoxybenzoic acid

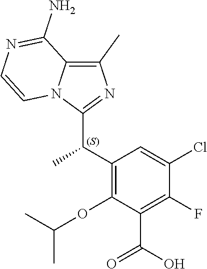

Ethyl (S)-3-(1-(8-amino-1-methylimidazo [1,5-a]pyrazin-3-yl)ethyl)-5-chloro-6-fluoro-2-isopropoxybenzoate (25.4 g, 58.5 mmol) in MeOH (100 mL) and H₂O (100 mL) was added NaOH (18.7 g, 468 mmol), the resulting solution was stirred at room temperature for overnight. After completion, the reaction solution was concentrated in vacuo to remove most of the MeOH, remaining solution was extracted with EtOAc (100 mL×2), the aqueous phase was adjusted pH to 2˜3, blown solid was precipitated, collected by filtration, dried in vacuo to give the product (15.8 g), the aqueous phase was extracted with DCM (100 mL×5), combined DCM phase was dried over MgSO4 and concentrated in vacuo to give another part of product (2.2 g), total yield (18 g, 75.6%). ¹H NMR (400 MHz, DMSO-d6) δ 7.78 (brs, 2H), 7.40-7.32 (m, 2H), 6.93 (d, J=5.3 Hz, 1H), 4.80 (q, J=7.0 Hz, 1H), 4.55 (dt, J=12.1, 6.0 Hz, 1H), 2.60 (s, 3H), 1.60 (d, J=7.0 Hz, 3H), 1.20 (d, J=6.0 Hz, 3H), 1.13 (d, J=6.0 Hz, 3H). LC-MS (M+H)+=407.1.

### (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide

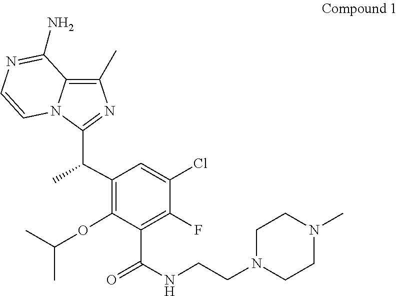 [SMILES: Cc1nc([C@@H](C)c2cc(Cl)c(F)c(C(=O)NCCN3CCN(C)CC3)c2OC(C)C)n2ccnc(N)c12]

This compound (93 mg, 72.1%) was prepared from 2-(4-methylpiperazin-1-yl)ethan-1-amine. ¹H NMR (400 MHz, DMSO-d6) δ 8.64-8.61 (t, 1H), 7.39-7.37 (d, J=8.8 Hz, 1H), 7.25-7.24 (d, J=5.2 Hz, 1H), 6.86-6.85 (d, J=4.8 Hz, 1H), 6.43 (brs, 2H), 4.80-4.74 (m, 1H), 4.52-4.46 (m, 1H), 3.41-3.28 (m, 4H), 2.56 (s, 3H), 2.43-2.18 (m, 8H), 2.14 (s, 3H), 1.59-1.57 (d, J=6.8 Hz, 3H), 1.19-1.18 (d, J=5.6 Hz, 3H), 1.10-1.08 (d, J=5.6 Hz, 3H). LC-MS (M+H)⁺=532.1. HPLC: 214 nm, 96.79%; 254 nm, 100%. RetTime in chiral-HPLC: 3.67 min.

### Preparation of Compound 2 (Fumarate)

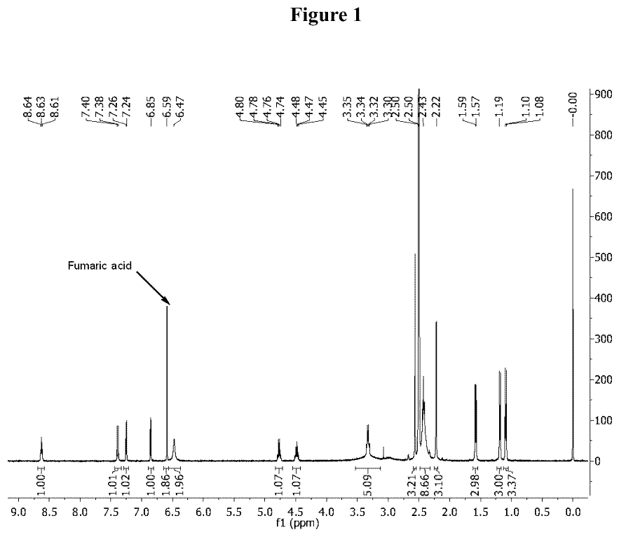

To a solution of (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide (5.0 g, the free base of Compound 1) in EtOH was added a solution of fumaric acid (970 mg) in EtOH. The mixture was stirred for 30 minutes. Then to the mixture was concentrated until about 24 g residue in the bottom. The resulting mixture was stirred at room temperature overnight, then the product was obtained (Compound 2). ¹H NMR spectrum showed the molar ratio of acid/free base was 1:1 (FIG. 1). ¹H NMR (400 MHz, dmso) δ 8.63 (t, J=5.5 Hz, 1H), 7.39 (d, J=8.6 Hz, 1H), 7.25 (d, J=5.1 Hz, 1H), 6.85 (d, J=5.0 Hz, 1H), 6.59 (s, 2H), 6.47 (brs, 2H), 4.77 (q, J=7.2 Hz, 1H), 4.52-4.44 (m, 1H), 3.33 q, J=6.3 Hz, 2H), 2.56 (s, 3H), 2.47-2.35 (m, 8H), 2.22 (s, 3H), 1.58 (d, J=7.0 Hz, 3H), 1.19 (d, J=6.0 Hz, 3H), 1.13-1.05 (m, 3H).

### Example 1: The Combination of a BTK Inhibitor and PI3Kδ Inhibitor in an MCL Xenograft Mouse Model

JeKo-1 cells are of mantle cell lymphoma (MCL) origin. These cells were cultured in RPMI1640 complete medium supplemented with 10% (v/v) fetal bovine serum, and 100 μg/mL of penicillin and streptomycin. NOD/SCID mice were pre-treated with cyclophosphamide (prepared in saline, 100 mg/kg, i.p.) and disulfiram (prepared in 0.8% Tween-80 in saline, 125 mg/kg, p.o., two hours after each dose of cyclophosphamide) once daily for two days.

On the day of implantation, aggregated cells were dispersed. Four hours later, the media was removed, and the cells were collected and re-suspended in cold (4° C.) DPBS with a final concentration of 1×10⁸ cells/mL. Re-suspended cells were placed on ice prior to inoculation. The right flank region of each mouse was cleaned with 75% ethanol prior to cell inoculation. Animals were then co-injected subcutaneously with 1×10⁷ JeKo-1 cells in 100 μL of cell suspension in the right front flank via a 26-gauge needle.

Animals were randomly divided into six groups with 10 mice per group. The groups consisted of vehicle group (0.5% (w/v) methylcellulose solution), 7.5 mg/kg BTK-1 (dissolved in 0.5% (w/v) methylcellulose solution), 12 mg/kg Compound 2 (dissolved in 0.5% (w/v) methylcellulose solution), 36 mg/kg Compound 2 (dissolved in 0.5% (w/v) methylcellulose solution), and the combination of Compound 2/BTK-1 (12 mg/kg Compound 2 and 7.5 mg/kg BTK-1), or (36 mg/kg Compound 2 and 7.5 mg/kg BTK-1), see Table 1. Compound 2 was (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide fumarate.

TABLE 1

| Exam- | Cell | Tumor | | | % |
|---|---|---|---|---|---|
| ple | Line | Type | Group | Dose(mg/Kg) | TGI |
| 1 | JeKo-1 | MCL | BTK-1 | 7.5 | 43 |
| | | | Compound 2 | 12 | 59 |
| | | | Compound 2 + | 12 + 7.5 | 75** |
| | | | BTK1 | | |
| | | | Compound 2 | 36 | 62 |
| | | | Compound 2 + | 36 + 7.5 | 84*** |
| | | | BTK1 | | |

**p < 0.01,
***p < 0.001

Both compounds were administered by oral gavage (p.o.) twice daily (BID). After implantation, primary tumor volume was measured in two dimensions using a caliper.

Individual body weight was recorded twice weekly, with mice being monitored daily for clinical signs of toxicity for the duration of the study. Mice were euthanized using carbon dioxide when their tumor volume reached 2,000 mm³, the tumor was ulcerated, or body weight loss exceeded 20%.

Tumor volume was calculated using the formula: V=0.5×(a×b²) where a and b are the long and short diameters of the tumor, respectively. Tumor growth inhibition (TGI) was calculated using the following formula: % TGI=100×[1−(treatedₜ/vehicleₜ)](treatedₜ=treated tumor volume at time t) and (vehicleₜ=vehicle tumor volume at time t)

The in vivo efficacy of BTK-1 and Compound 2 combination was examined in the JeKo-1 xenograft model. Treatment with BTK-1 as a single agent was shown to be active in this model, with 43% TGI on day 21. Compound 2 as a single agent, had 59% TGI on day 21 of treatment at a dose of 12 mg/kg and 62% TGI on day 21 at 36 mg/kg(mpk). The combination of these two agents induced 75% TGI on day 21 with a 12 mg/kg Compound 2/7.5 mg/kg BTK-1 combination and 84% TGI on day 21 with 36 mg/kg Compound 2/7.5 mg/kg BTK-1 which was significantly more efficacious than either single agent. This result is shown in Table 1.

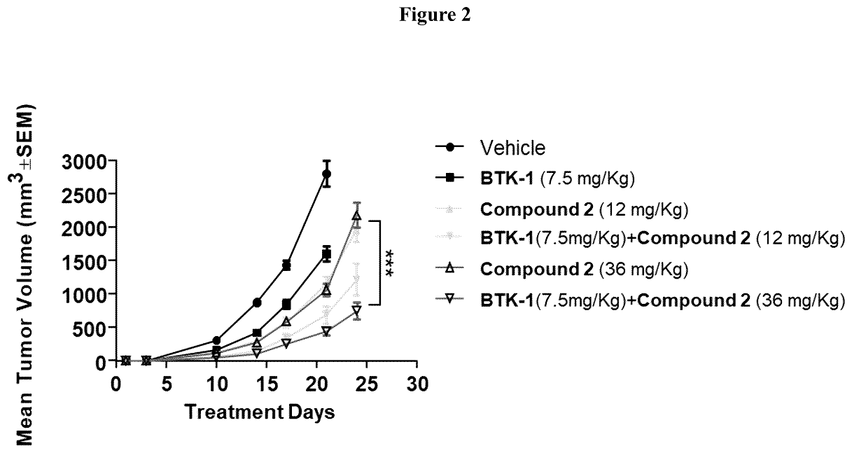

The JeKo-1 mouse model was treated for 21 days with BTK-1 at 7.5 mg/kg, for 24 days with Compound 2 at 12 mg/kg or Compound 2 at 36 mg/kg, each compound was administered as a single agent. The combination of BTK-1 and Compound 2 was administered at BTK-1 (7.5 mg/kg) and Compound 2 (12 mg/kg) or at BTK-1 (7.5 mg/kg) and Compound 2 (36 mg/kg). This result is shown graphically in FIG. 2. The highest dose of the combination (combination of 7.5 mg/kg BTK-1/36 mg/kg Compound 2) was the most efficacious and this result is shown in FIG. 2. The combination was well tolerated in general, and no loss of body weight was noted (data not shown).

### Example 2: The Combination of a BTK Inhibitor and PI3Kδ Inhibitor in an MCL Xenograft Mouse Model

MINO cells are of MCL origin. The MINO cells were cultured in RPMI1640 complete medium supplemented with 10% (v/v) fetal bovine serum, and 100 μg/mL of penicillin and streptomycin. On the day of implantation, aggregated cells were dispersed. Four hours later, media was removed, and the cells were collected as described above. Cells were re-suspended in cold (4° C.) PBS with a final concentration of 1×10⁸ cells/mL. Re-suspended cells were placed on ice prior to inoculation. The right flank region of each mouse (NOD/SCID) was cleaned with 75% ethanol prior to cell inoculation. Animals were then injected subcutaneously with 1×10⁷ MINO cells in 100 μl of cell suspension in the right front flank via a 26-gauge needle.

On day 1 after inoculation, animals were randomly divided into 6 groups with 10 mice per group according to the inoculation order. The groups consisted of vehicle group (0.5% (w/v) methylcellulose solution), 7.5 mg/kg BTK-1 (dissolved in 0.5% (w/v) methylcellulose solution), 12 mg/kg Compound 2 (dissolved in 0.5% (w/v) methylcellulose solution), 36 mg/kg Compound 2 (dissolved in 0.5% (w/v) methylcellulose solution) and the combination of Compound 2/BTK-1 (12 mg/kg Compound 2 and 7.5 mg/kg BTK-1), or (36 mg/kg Compound 2 and 7.5 mg/kg BTK-1), see Table 2. The compounds were administered by oral gavage (p.o.) twice daily (BID). After implantation, primary tumor volume was measured in two dimensions using a caliper. Compound 2 was (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide fumarate.

TABLE 2

| Exam- | Cell | Tumor | | | % |
|---|---|---|---|---|---|
| ple | Line | Type | Group | Dose(mg/Kg) | TGI |
| 2 | MINO | MCL | BTK-1 | 7.5 | 22 |
| | | | Compound 2 | 12 | 26 |
| | | | Compound 2 + | 12 + 7.5 | 75 |
| | | | BTK1 | | |
| | | | Compound 2 | 36 | 36 |
| | | | Compound 2 + | 36 + 7.5 | 66 |
| | | | BTK1 | | |

Mice were euthanized using carbon dioxide when their tumor volume reached 2,000 mm³ after twice measurements, the tumor was ulcerated, or body weight loss exceeded 20%.

Tumor volume was calculated using the formula: V=0.5×(a×b²) where a and b are the long and short diameters of the tumor, respectively. Tumor growth inhibition (TGI) was calculated using the following formula: % TGI=100×[1−(treatedₜ/vehicleₜ)](treatedₜ=treated tumor volume at time t) and (vehicleₜ=vehicle tumor volume at time t)

On day 24, treatment with BTK-1 resulted in only 22% tumor growth inhibition (TGI). Treatment with Compound 2, as a single agent had 26% TGI on day 24 of treatment at a concentration of 12 mg/kg and 36% TGI on day 24 at 36 mg/kg. The combination of these two agents produced 75% TGI on day 24 with a 12 mg/kg Compound 2/7.5 mg/kg BTK-1 combination and 66% TGI on day 24 with 36 mg/kg Compound 2/7.5 mg/kg BTK-1 which was significantly more efficacious than either single agent and these results are shown in Table 2.

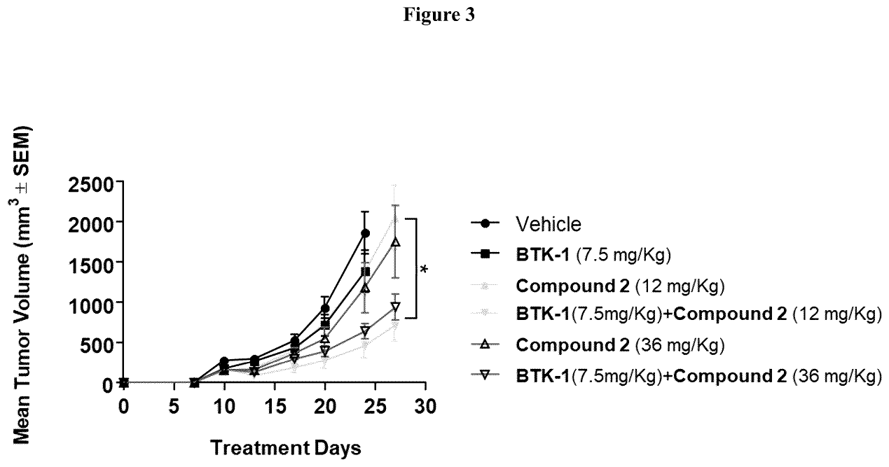

The MINO mouse model was treated for 24 days with BTK-1 at 7.5 mg/kg, for 27 days with Compound 2 at 12 mg/kg or Compound 2 at 36 mg/kg, with each compound administered as a single agent. The combination of BTK-1 and Compound 2 was administered at BTK-1 (7.5 mg/kg) and Compound 2 (12 mg/kg) or at BTK-1 (7.5 mg/kg) and Compound 2 (36 mg/kg). The combination of BTK-1 and Compound 2 at 12 mg/kg was the most efficacious dose in this model, and this result is shown in FIG. 3. The combination was well tolerated at the lower dose as well as the higher 36 mg/kg dose with no loss of body weight noted.

### Example 3: The Combination of a BTK Inhibitor and PI3Kδ Inhibitor in a DLBCL Xenograft Mouse Model

TMD8 cells are of DLBCL origin. TMD8 cells were cultured in RPMI1640 complete medium supplemented with 10% (v/v) fetal bovine serum, and 100 μg/mL of penicillin and streptomycin. On the day of implantation, aggregated cells were dispersed. Four hours later, media was removed, and the cells were collected as described above. Cells were re-suspended in cold (4° C.) PBS and same volume of matrigel was added to give a final concentration of 5×10⁷ cells/mL for TMD8. Re-suspended cells were placed on ice prior to inoculation. The right flank region of each mouse (NOD/SCID) was cleaned with 75% ethanol prior to cell inoculation. Animals were then injected subcutaneously with 1×10⁷ TMD8 cells in 200 μl of cell suspension in the right front flank via a 26-gauge needle.

On day 15 after inoculation, animals were randomly divided into 8 groups with 10 mice per group according to the tumor volume. The groups consisted of vehicle group (0.5% (w/v) methylcellulose solution), 2.5 mg/kg BTK-1 (dissolved in 0.5% (w/v) methylcellulose solution), 10 mg/kg Compound 1 (dissolved in 0.5% (w/v) methylcellulose solution), 30 mg/kg Compound 1 (dissolved in 0.5% (w/v) methylcellulose solution), 100 mg/kg Compound 1 (dissolved in 0.5% (w/v) methylcellulose solution) and the combination of Compound 1/BTK-1 (10 mg/kg Compound 1 and 2.5 mg/kg BTK-1), (30 mg/kg Compound 1 and 2.5 mg/kg BTK-1), or (100 mg/kg Compound 1 and 2.5 mg/kg BTK-1), see Table 3. BTK-1 and Compound 1 were administered by oral gavage (p.o.) twice daily (BID). After implantation, primary tumor volume was measured in two dimensions using a caliper. Compound 1 was (S)-3-(1-(8-amino-1-methylimidazo[1,5-a] pyrazin-3-yl)ethyl)-5-chloro-6-fluoro-2-isopropoxy-N-(2-(4-methylpiperazin-1-yl)ethyl)benzamide.

TABLE 3

| Exam- | Cell | Tumor | | | % |
|---|---|---|---|---|---|
| ple | Line | Type | Group | Dose(mg/Kg) | TGI |
| 3 | TMD8 | DLBCL | BTK-1 | 2.5 | 68 |
| | | | Compound 1 | 10 | 27 |
| | | | Compound 1 + | 10 + 2.5 | 77 |
| | | | BTK1 | | |
| | | | Compound 1 | 30 | 26 |
| | | | Compound 1 + | 30 + 2.5 | 95*** |
| | | | BTK1 | | |
| | | | Compound 1 | 100 | 43 |
| | | | Compound 1 + | 100 + 2.5 | 101* |
| | | | BTK1 | | |

**p < 0.01,
***p < 0.001

Individual body weight was recorded twice weekly, with mice being monitored daily for clinical signs of toxicity for the duration of the study. Mice were euthanized using carbon dioxide when their tumor volume reached 2,000 mm³ after twice measurements, the tumor was ulcerated, or body weight loss exceeded 20%.

Tumor volume was calculated using the formula: V=0.5×(a×b²) where a and b are the long and short diameters of the tumor, respectively. Tumor growth inhibition (TGI) was calculated using the formula in the above examples.

On day 14 post treatment, treatment with BTK-1 as a single agent resulted in only 68% tumor growth inhibition (TGI). Compound 1 as a single agent, had 27% TGI on day 14 of treatment at a concentration of 10 mg/kg, a 26% TGI with a 30 mg/kg dose and a 43% TGI with a 100 mg/kg dose. The combination of BTK-1 and Compound 1 with a 10 mg/kg Compound 1/2.5 mg/kg BTK-1 combination resulted in 77% TGI on day 14, 95% TGI with 30 mg/kg Compound 1/2.5 mg/kg BTK-1 combination. Finally, the combination at 100 mg/kg Compound 1/2.5 mg/kg BTK-1 resulted in and 101% TGI. These results are shown in Table 3.

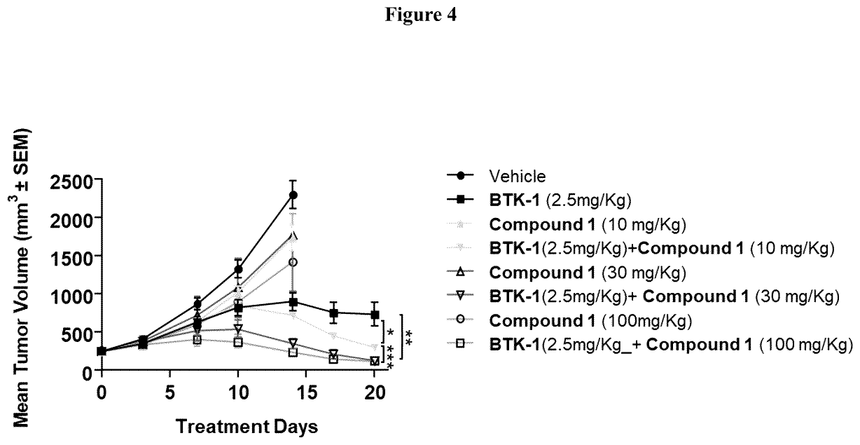

The TMD8 mouse model was treated for 20 days with BTK-1 at 2.5 mg/kg, Compound 1 at 10 mg/kg, Compound 1 at 30 mg/kg or Compound 1 at 100 mg/kg, with each respective compound administered as a single agent. The combination of BTK-1 and Compound 1 was administered at BTK-1 (2.5 mg/kg) and Compound 1 (10 mg/kg), at BTK-1 (2.5 mg/kg) and Compound 1 (30 mg/kg) or BTK-1 (2.5 mg/kg) and Compound 1 (100 mg/kg). The combination of BTK-1 (2.5 mg/kg) and Compound 1 at the 100 mg/kg dose was the most efficacious in this model, and this result is shown in FIG. 4. The combination was well tolerated at all doses with no loss of body weight.

### Example 4: The Combination of a BTK Inhibitor and PI3Kδ Inhibitor in a DLBCL Xenograft Mouse Model

Farage cells are of DLBCL origin. Farage cells were cultured in RPMI1640 complete medium supplemented with 10% (v/v) fetal bovine serum, and 100 μg/mL of penicillin and streptomycin. On the day of implantation, aggregated cells were dispersed. Four hours later, media was removed and the cells were collected as described above. Cells were re-suspended in cold (4° C.) PBS and same volume of matrigel was added to give a final concentration of 1.5×10⁷ cells/mL for Farage. Re-suspended cells were placed on ice prior to inoculation. The right flank region of each mouse (NCG) was cleaned with 75% ethanol prior to cell inoculation. Animals were then injected subcutaneously with 3×10⁶ Farage cells in 100 μl of cell suspension in the right front flank via a 26-gauge needle.

The animals were randomly divided into 6 groups with 10 mice per group. The groups consisted of vehicle group (0.5% (w/v) methylcellulose solution), 7.5 mg/kg BTK-1 (dissolved in 0.5% (w/v) methylcellulose solution), 12 mg/kg Compound 2 (dissolved in 0.5% (w/v) methylcellulose solution), 36 mg/kg Compound 2 (dissolved in 0.5% (w/v) methylcellulose solution), and the combination of Compound 2 and BTK-1 (12 mg/kg and 7.5 mg/kg, respectively), or (36 mg/kg and 7.5 mg/kg, respectively), see Table 4. BTK-1 and Compound 2 were administered as a combination by oral gavage (p.o.) twice daily (BID). After implantation, primary tumor volume was measured in two dimensions using a caliper. Compound 2 was (S)-3-(1-(8-amino-1-methylimidazo [1,5-a] pyrazin-3-yl) ethyl)-5-chloro-6-fluoro-2-isopropoxy-N-(2-(4-methylpiperazin-1-yl) ethyl) benzamide fumarate.

TABLE 4

| Exam- | Cell | Tumor | | | % |
|---|---|---|---|---|---|
| ple | Line | Type | Group | Dose(mg/Kg) | TGI |
| 4 | Farage | DLBCL | BTK-1 | 7.5 | 43 |
| | | | Compound 2 | 12 | 40 |
| | | | Compound 2 + | 12 + 7.5 | 64 |
| | | | BTK1 | | |
| | | | Compound 2 | 36 | 52 |
| | | | Compound 2 + | 36 + 7.5 | 76 |
| | | | BTK1 | | |

Individual body weight was recorded twice weekly, with mice being monitored daily for clinical signs of toxicity for the duration of the study. Mice were euthanized using carbon dioxide when their tumor volume reached 2,000 mm³ after twice measurements, the tumor was ulcerated, or body weight loss exceeded 20%.

Tumor volume was calculated using the formula: V=0.5×(a×b²) where a and b are the long and short diameters of the tumor, respectively. Tumor growth inhibition (TGI) was calculated using the formula in the above examples.

On day 25, treatment with BTK-1 alone resulted in only 43% TGI. Compound 2, as a single agent had 40% TGI on day 25 of treatment at a concentration of 12 mg/kg and a 52% TGI on day 25 at 36 mg/kg. The combination of BTK-1 and Compound 2 induced 64% TGI on day 25 with a 12 mg/kg Compound 2/7.5 mg/kg BTK-1 combination, 76% TGI on day 25 with 36 mg/kg Compound 2/7.5 mg/kg BTK-1. This is shown in Table 4.

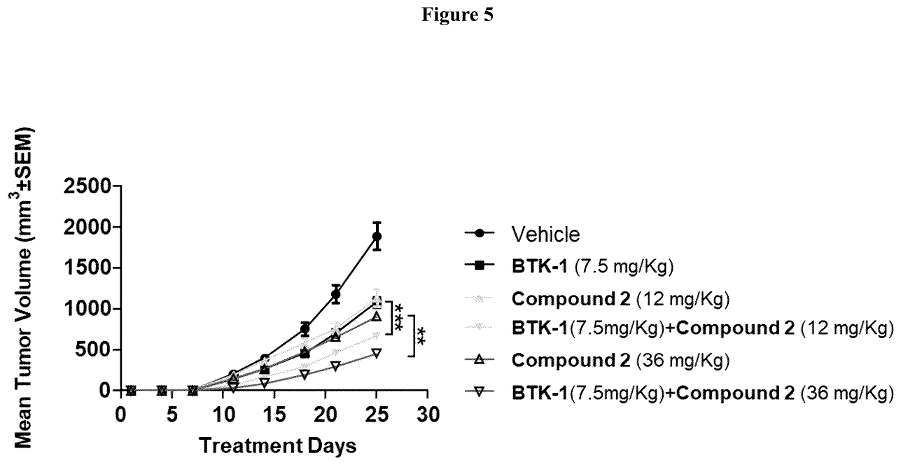

The Farage mouse model was treated for 25 days with BTK-1 at 7.5 mg/kg or Compound 2 at 12 mg/kg BID, with each compound administered as a single agent. The combination of BTK-1 and Compound 2 was administered at BTK-1 (7.5 mg/kg) and Compound 2 (12 mg/kg) or BTK-1 (7.5 mg/kg) and Compound 2 (36 mg/kg). This result is shown graphically in FIG. 5. The combination was well tolerated at the lower dose as well as the higher 36 mg/kg dose with no loss of body weight noted.

### Example 5 Clinical Trials of a BTK Inhibitor in Combination with PI3Kδ Inhibitor

The purpose of the trials is to evaluate the safety and effectiveness of BTK-1 and Compound 2, in patients with Mature B-cell malignancies such as MZL, FL, MCL, or DLBCL.

A Dose Escalation Phase to Determine the MTD/RP2D of Compound 2 in Combination with BTK-1

Compound 2 was administered orally QD at a dose level lower less than RP2D identified in monotherapy dose escalation and RP2D in combination with BTK-1 160 mg (2*80 mg capsules) administered orally twice daily (BID).

A Dose Expansion Phase for Evaluation of Compound 2 in Combination with BTK-1 at the RP2D in Patients with R/R FL, R/R MCL and R/R DLBCL

Compound 2 was administered orally QD at RP2D in combination with BTK-1 160 mg (2*80 mg capsules) administered orally BID.

Patients with MZL, FL, MCL, or DLBCL must have at least one bi-dimensionally measurable nodal lesion >1.5 cm in longest diameter or extranodal lesion that is >1 cm in longest diameter by computed tomography (CT) scan or magnetic resonance imaging (MRI), as defined by the Lugano Classification.

### Key Exclusion Criteria:

History of allogeneic stem-cell transplantation, and
Prior exposure to PI3K inhibitor and/or BTK inhibitor.

Any approved anticancer therapy, including hormonal therapy, or any investigational agent or participation in another clinical study with therapeutic intent within 14 days or 5-half lives which is shorter before the first dose.

Known human immunodeficiency virus (HIV) infection, or serologic status reflecting active viral hepatitis B (HBV) or viral hepatitis C (HCV) infection as follows:
HBsAg (+), or
HBcAb (+) and HBV DNA detected, or
Presence of HCV antibody. Patients with the presence of HCV antibody are eligible if HCV ribonucleic acid (RNA) is undetectable.

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

1. A method for the treatment or delay of progression of cancer in a subject, comprising administering to the subject in need thereof a therapeutically effective amount of (S)-7-(1-acryloylpiperidin-4-yl)-2-(4-phenoxyphenyl)-4,5,6,7-tetra-hydropyrazolo[1,5-a]pyrimidine-3-carboxamide, or a pharmaceutically acceptable salt thereof, in combination with a therapeutically effective amount of a PI3Kδ inhibitor.

2. The method of claim 1, wherein the PI3Kδ inhibitor is selected from the group consisting of Idelalisib, Copanlisib, Duvelisib, Umbralisib, Leniolisib, Parsaclisib, AMG-319, ME-401, Tenalisib, Linperlisib, Seletalisib, Nemiralisib, KA-2237, SF-1126, HMPL-689, ACP-319, SHC-014748M, AZD-8154, PI3065, and (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide, or a pharmaceutically acceptable salt thereof.

3. The method of claim 2, wherein the PI3Kδ inhibitor is (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide, or a pharmaceutically acceptable salt thereof.

4. The method of claim 3, wherein the pharmaceutically acceptable salt is fumarate.

5. The method of claim 1, wherein the cancer is hematologic cancer.

6. The method of claim 5, wherein the hematologic cancer is leukemia, lymphoma, myeloma, non-Hodgkin's lymphoma (NHL), Hodgkin's lymphoma (HL), or B-cell malignancy.

7. The method of claim 6, wherein the B-cell malignancy is chronic lymphocytic leukemia (CLL), small lymphocytic lymphoma (SLL), follicular lymphoma (FL), mantle cell lymphoma (MCL), marginal zone lymphoma (MZL), Waldenstrom macroglobulinemia (WM), Hairy cell leukemia (HCL), Burkitt's-like leukemia (BL), B cell prolymphocytic leukemia (B-PLL), diffuse large B-cell lymphoma (DLBCL), germinal center B-cell diffuse large B-cell lymphoma (GCB-DLBCL), non-germinal center B-cell diffuse large B-cell lymphoma (non-GCB DLBCL), DLBCL with undetermined subtype, primary central nervous system lymphoma (PCNSL), or secondary central nervous system lymphoma (SCNSL) of breast or testicular origin.

8. The method of claim 7, wherein the diffuse large B-cell lymphoma (DLBCL) is activated B-cell diffuse large B-cell lymphoma (ABC-DLBCL), GCB-DLBCL or non-GCB DLBCL.

9. The method of claim 6, wherein the B-cell malignancy is a resistant B-cell malignancy.

10. The method of claim 9, wherein the resistant B-cell malignancy is chronic lymphocytic leukemia (CLL), small lymphocytic lymphoma (SLL), follicular lymphoma (FL), mantle cell lymphoma (MCL), marginal zone lymphoma (MZL), Waldenstrom macroglobulinemia (WM), Hairy cell leukemia (HCL), Burkitt's-like leukemia (BL), B cell prolymphocytic leukemia (B-PLL), diffuse large B cell lymphoma (DLBCL), germinal center B-cell diffuse large B-cell lymphoma (GCB-DLBCL), non-germinal center B-cell diffuse large B-cell lymphoma (non-GCB DLBCL), DLBCL with undetermined subtype, primary central nervous system lymphoma (PCNSL), or secondary central nervous system lymphoma (SCNSL) of breast or testicular origin.

11. The method of claim 10, wherein the resistant B-cell malignancy is diffuse large B-cell lymphoma (DLBCL).

12. The method of claim 11, wherein the resistant DLBCL is activated B-cell diffuse large B-cell lymphoma (ABC-DLBCL), GCB-DLBCL or non-GCB DLBCL.

13. The method of claim 1, wherein the cancer is a sarcoma, or carcinoma.

14. The method of claim 13, wherein the cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma.

15. The method of claim 14, wherein the cancer is a resistant cancer.

16. The method of claim 15, wherein the resistant cancer is selected from bladder cancer, breast cancer, colon cancer, gastroenterological cancer, kidney cancer, lung cancer, ovarian cancer, pancreatic cancer, prostate cancer, proximal or distal bile duct cancer, or melanoma.

17. The method of claim 1, wherein the BTK inhibitor is administered at a dose from 50 mg to 320 mg, 50 mg, 100 mg, 150 mg, 200 mg, 250 mg, 300 mg, 350 mg, 400 mg, 450 mg, 500 mg, 550 mg or 600 mg QD or 20 mg, 40 mg, 60 mg, 80 mg, 100 mg, 120 mg, 140 mg, 160 mg, 180 mg, 200 mg, 220 mg, 240 mg, 260 mg, 280 mg, 300 mg or 320 mg BID.

18. The method of claim 17, wherein the BTK inhibitor is administered at a dose of 320 mg QD or 160 mg BID.

19. The method of claim 1, wherein the PI3Kδ inhibitor is administered at a dose from 20 mg to 600 mg QD, 20-120 mg QD, 40-250 mg QD, 200-400 mg QD, 400-600 mg QD, s 20 mg QD, 40 mg QD, 60 mg QD, 80 mg QD, 100 mg QD, 120 mg QD, 140 mg QD, 160 mg QD, 180 mg QD, 200 mg QD, 220 mg QD, 240 mg QD, 260 mg QD, 280 mg QD, 300 mg QD, 320 mg QD, 340 mg QD, 360 mg QD, 380 mg QD, 400 mg QD, 420 mg QD, 440 mg QD, 460 mg QD, 480 mg QD, 500 mg QD, 520 mg QD, 540 mg QD, 560 mg QD, or 580 mg QD.

20. The method of claim 1, wherein the PI3Kδ inhibitor is administered at a dose from 20 mg to 320 mg BID, 20 mg BID, 40 mg BID, 60 mg BID, 80 mg BID, 100 mg BID, 120 mg BID, 140 mg BID, 160 mg BID, 180 mg BID, 200 mg BID, 220 mg BID, 240 mg BID, 260 mg BID, 280 mg BID, 300 mg BID or 320 mg BID.

21. The method of claim 1, wherein the dosage of the PI3Kδ inhibitor is between 5 mg to 80 mg per capsule, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, or 80 mg per capsule.

22. A pharmaceutical composition for use in the treatment or delay of progression of cancer, comprising administering to the subject in need thereof a therapeutically effective amount of (S)-7-(1-acryloylpiperidin-4-yl)-2-(4-phenoxyphenyl)-4,5,6,7-tetra-hydropyrazolo[1,5-a]pyrimidine-3-carboxamide (BTK-1), or a pharmaceutically acceptable salt thereof, in combination with a therapeutically effective amount of a PI3Kδ inhibitor or a pharmaceutically acceptable salt thereof.

23. The pharmaceutical composition of claim 21, wherein the PI3Kδ inhibitor is selected from the group consisting of: Idelalisib, Copanlisib, Duvelisib, Umbralisib, Leniolisib, Parsaclisib, AMG-319, ME-401, Tenalisib, Linperlisib, Seletalisib, Nemiralisib, KA-2237, SF-1126, HMPL-689, ACP-319, SHC-014748M, AZD-8154, PI3065, and (S)-3-(1-(8-amino-1-methylimidazo [1,5-a]pyrazin-3-yl)ethyl)-5-chloro-6-fluoro-2-isopropoxy-N-(2-(4-methylpiperazin-1-yl)ethyl)benzamide, or a pharmaceutically acceptable salt thereof.

24. The pharmaceutical composition of claim 22, wherein the PI3Kδ inhibitor is (S)-3-(1-(8-amino-1-methylimidazo [1,5-a]pyrazin-3-yl)ethyl)-5-chloro-6-fluoro-2-isopropoxy-N-(2-(4-methylpiperazin-1-yl)ethyl)benzamide, or a pharmaceutically acceptable salt thereof.

25. The pharmaceutical composition of claim 23, wherein the pharmaceutically acceptable salt is fumarate.

26. A pharmaceutical combination for use in the treatment or delay of progression of cancer, comprising administering to the subject in need thereof a therapeutically effective amount of (S)-7-(1-acryloylpiperidin-4-yl)-2-(4-phenoxyphenyl)-4,5,6,7-tetra-hydropyrazolo[1,5-a]pyrimidine-3-carboxamide (BTK-1), or a pharmaceutically acceptable salt thereof, in combination with a therapeutically effective amount of a PI3Kδ inhibitor or a pharmaceutically acceptable salt thereof.

27. The pharmaceutical combination for use of claim 25, wherein the PI3Kδ inhibitor is selected from the group consisting of: Idelalisib, Copanlisib, Duvelisib, Umbralisib, Leniolisib, Parsaclisib, AMG-319, ME-401, Tenalisib, Linperlisib, Seletalisib, Nemiralisib, KA-2237, SF-1126, HMPL-689, ACP-319, SHC-014748M, AZD-8154, PI3065 and (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide, or a pharmaceutically acceptable salt thereof.

28. The pharmaceutical combination for use of claim 26, wherein the PI3Kδ inhibitor is (S)-3-(1-(8-amino-1-methylimidazo[1,5-a]pyrazin-3-yl)ethyl)-5-chloro-6-fluoro-2-isopropoxy-N-(2-(4-methylpiperazin-1-yl)ethyl)benzamide, or a pharmaceutically acceptable salt thereof.

29. The pharmaceutical combination for use of claim 27, wherein the pharmaceutically acceptable salt is fumarate.

30. The pharmaceutical combination for use of claim 25, wherein the cancer is selected from leukemia, lymphoma, myeloma, non-Hodgkin's lymphoma (NHL), Hodgkin's lymphoma (HL), or B-cell malignancy.

31. The pharmaceutical combination for use of claim 29, wherein the B-cell malignancy is chronic lymphocytic leukemia (CLL), small lymphocytic lymphoma (SLL), follicular lymphoma (FL), mantle cell lymphoma (MCL), marginal zone lymphoma (MZL), Waldenstrom macroglobulinemia (WM), Hairy cell leukemia (HCL), Burkitt's-like leukemia (BL), B cell prolymphocytic leukemia (B-PLL), diffuse large B cell lymphoma (DLBCL), germinal center B-cell diffuse large B-cell lymphoma (GCB-DLBCL), non-germinal center B-cell diffuse large B-cell lymphoma (non-GCB DLBCL), DLBCL with undetermined subtype, primary central nervous system lymphoma (PCNSL), secondary central nervous system lymphoma (SCNSL) of breast or testicular origin, or a combination of two or more thereof.

32. The pharmaceutical combination for use of claim 30, wherein the DLBCL is activated B-cell diffuse large B-cell lymphoma (ABC-DLBCL), GCB-DLBCL or non-GCB DLBCL.

33. The pharmaceutical combination for use of claim 29, wherein the B-cell malignancy is a resistant B-cell malignancy.

34. The pharmaceutical combination for use of claim 25, wherein the cancer is a sarcoma, or carcinoma.

35. The pharmaceutical combination for use of claim 33, wherein the cancer is selected from bladder cancer, breast cancer, colon cancer, gastroenterological cancer, kidney cancer, lung cancer, ovarian cancer, pancreatic cancer, prostate cancer, proximal or distal bile duct cancer, and melanoma.

36. The pharmaceutical combination for use of claim 33, wherein the cancer is a BTK inhibitor resistant cancer.
